SEQUENCE LISTING (1 sequence)

SEQ ID NO: 1 - protein, 265 aa, Sepioteuthis lessioniana
MSSFMDPMHYDGMGMSHSKSGDFSHNCMRSFHKSQRDVMRRDIMGKSNKNRRFGNLMEPMSRMTMDFHGRLIDSQGRIVDPGHYFAMDDHYMENERFLYPHDMIRNRHGMYGFMQGDNVYGNNMHRGMFADGMYRDMHHSGMHPSGYMHGGSMQNRPMMYMQGRYPDDSYFMNYHPSMSDPPVIMHSHYNDHEGRHGMYGRPSESYGSHGRHGDAHSMPRRVSDTHSPQRRPSESHIIQVRPEGGSSRKTSRAKLFPDDKNTDSA

(19) United States
(12) Patent Application Publication
(10) Pub. No.: US 2024/0400624 A1
(43) Pub. Date: Dec. 5, 2024

(54) A RECOMBINANT REFLECTIN NANOPARTICLE

(71) Applicant: NANYANG TECHNOLOGICAL UNIVERSITY, Singapore (SG)

(72) Inventors: Jun Jie LOKE, Singapore (SG); Ali Gilles Tchenguise MISEREZ, Singapore (SG)

(21) Appl. No.: 18/700,680

(22) PCT Filed: Oct. 13, 2022
(86) PCT No.: PCT/SG2022/050732

§ 371 Date: Apr. 11, 2024

(30) Foreign Application Priority Data
Oct. 13, 2021 (SG) 10202111370U

(51) Int. Cl. C07K 14/435; A61K 8/02; A61K 8/64; A61Q 19/00

(52) U.S. Cl. CPC C07K 14/43504; A61K 8/0241; A61K 8/64; A61Q 19/00; A61K 2800/412

(57) ABSTRACT

There is provided a reflectin polypeptide comprising an amino acid sequence that shares at least 70% sequence identity or at least 80% sequence homology with the amino acid sequence as set forth in SEQ ID NO: 1, a nucleic acid molecule encoding the reflectin polypeptide, a host cell comprising the nucleic acid molecule; a recombinant reflectin nanoparticle comprising the said reflectin polypeptide; a method of synthesizing the recombinant reflectin nanoparticle, a substrate surface-functionalized with the recombinant reflectin nanoparticle; and a skincare product comprising the recombinant reflectin nanoparticle.

### CROSS-REFERENCE TO RELATED APPLICATION

This application claims the benefit of priority of Singapore Patent Application No. 10202111370U, filed 13 Oct. 2021, the contents of it being hereby incorporated by reference in its entirety for all purposes.

### REFERENCE TO AN ELECTRONIC SEQUENCE LISTING

The contents of the electronic sequence listing (P123984-sequence_listing.xml; Size: 1,979 bytes; and Date of Creation: Oct. 13, 2022) is herein incorporated by reference in its entirety.

### TECHNICAL FIELD

An aspect of the disclosure relates to a reflectin polypeptide. Another aspect of the disclosure relates to a recombinant reflectin nanoparticle. Another aspect of the disclosure relates to a recombinant reflectin nanoparticle immobilized on a surface and a method of producing the recombinant reflectin nanoparticle and immobilizing it on the surface. Another aspect of the disclosure relates to a skincare product comprising the recombinant reflectin nanoparticle.

### BACKGROUND

Cephalopods (octopus, squid and cuttlefish) are masters of camouflage of the animal kingdom. They use metachrosis to dynamically control the morphology of dermal cells-chromatophores and iridophores—to regulate body colouration and patterns. In fact, the paralarvae of Sepioteuthis lessioniana (Bigfin reef squid) are capable of producing highly complex yet mesmerising body patterns from the moment they hatch. The use of reflective tissues to convey signals is prevalent in nature and typically serves important survival functions to deter predators, capture prey, and for signalling. These iridescent light reflective-refractive structures often rely on Bragg reflectors, making use of periodic spacing of photonic crystals and thin-film constructive interference. Such tissues can typically be found in butterfly wings, peacock feathers, or in specialised tapetum lucidum reflective tissues found in the eyes of certain vertebrates.

Squids in the Loliginidae family (including Sepioteuthis lessionia) possess the unique capability to dynamically modulate the iridescent properties of their skin by tuning and controlling the internal assembly and periodicity of Bragg-like reflector platelets located within iridophores, which are entirely made of proteins called reflectins. This is in contrast to reflector platelets of other animals which are comprised of purine crystals. Previous studies have demonstrated that these dynamic photonic characteristics are regulated by phosphorylation/dephosphorylation of condensed reflectin nanoparticles in the reflector platelets. Phosphorylation of multiple tyrosine (Tyr), serine (Ser), or threonine (Thr) residues, which is controlled by the neurotransmitter acetylcholine (ACh), quickly imparts negative charges to the positively-charged reflectins, resulting in charge neutralization and a decrease in nanoparticle size, eventually leading to a blueshift in emitted wavelength. This results in dynamic iridescence, an angle- and wavelength-dependent reflection that gives rise to a range of vivid colours.

The remarkable dynamic camouflage ability of cephalopods arises from precisely orchestrated structural changes within their chromatophores and iridophores photonic cells. This mesmerizing colour display remains unmatched in synthetic coatings and is regulated by swelling/de-swelling of reflectin nanoparticles, which alters platelets dimensions in iridophores to control photonic patterns according to Bragg's law.

Initial research into reflectin A1 suggested that the formation and self-assembly of reflectin nanoparticles occurred due to the presence of the repeated motifs. However, the studies did not use full sequence reflectin protein, and instead only worked on mutated and truncated sequences.

Since there appear to be various fields of application for reflectins, there is a need for the provision of said reflectins.

### SUMMARY

In a first aspect, there is provided a reflectin polypeptide comprising an amino acid sequence that shares at least 70% sequence identity or at least 80% sequence homology with the amino acid sequence as set forth in SEQ ID NO:1, wherein said reflectin polypeptide substantially retains the activity of reflectin B1 (SEQ ID NO:1).

In a second aspect, there is provided a nucleic acid molecule encoding the reflectin polypeptide as described above.

In a third aspect, there is provided a host cell comprising the nucleic acid molecule as described above, wherein the host cell is a bacterial cell.

In a fourth aspect, there is provided a recombinant reflectin nanoparticle comprising a reflectin polypeptide as described above.

In a fifth aspect, there is provided a method of synthesizing a recombinant reflectin nanoparticle as described herein.

In a sixth aspect, there is provided a substrate surface-functionalized with a recombinant reflectin nanoparticle as described herein.

In a seventh aspect, there is provided a method of immobilizing a recombinant reflectin nanoparticle as described above on a substrate.

In an eighth aspect, there is provided a skincare product comprising a recombinant reflectin nanoparticle as described above.

### BRIEF DESCRIPTION OF THE DRAWINGS

The invention will be better understood with reference to the detailed description when considered in conjunction with the non-limiting examples and the accompanying drawings, in which:

FIG. 1 shows a one step purification of unconjugated S. lessoniana B1 reflectin (SIRF-B1) using strong cation exchange chromatography, wherein FIG. 1A shows a chromatogram of SIRF-B1 crude extract purification at pH 6.0 with protein peaks between the 21^{st} to 27^{th} minute; FIG. 1B shows the sodium dodecyl sulfate-polyacrylamide gel electrophoresis (SDS-PAGE) of the pooled protein peaks indicating a high purity of the monomer and its dimer; FIG. 1C shows a matrix assisted laser desorption ionization-time of flight (MALDI-ToF) mass spectrum confirming the molecular weight and purity of the collected fraction; FIG. 1D shows an average particle size measured by dynamic light scattering (DLS) of SIRF-B1 nanoparticles assembled against different concentrations of acetonitrile (ACN) before and after dibenzocyclooctyne (DBCO) conjugation. The error bar represents the final nanoparticle size averaged across different batches of samples produced (n=50), and not the size dispersity of an individual sample; colour scheme of visible wavelength from 380-780 nm was used as the reference to compare the equivalent nanoparticle size.

FIG. 2 shows DLS monitoring and schematic illustration of the different stages of DBCO-SIRF-B1 nanoparticles growth, wherein FIG. 2A shows a DLS scattered plot and curve fitting of 360 nm and 660 nm DBCO-SIRF-B1 nanoparticles as a function of time, illustrating that the growth is rapid and saturated within a few minutes, with particle size ripening in thirty minutes; FIG. 2B shows the growth of 660 nm nanoparticles and its respective polydispersity index (PDI), divided into four steps; FIG. 2C is a schematic representation of the nanoparticle growth illustrated in steps 1-4. Step 1: The growth is initiated by the addition of DBCO-Sulfo-NHS ester. Step 1: nanoparticles initially coalesce to form larger anisotropic nanoparticles that increased the PDI. Step 2: largest achievable nanoparticle size with significantly low PDI due to an equilibrium between the inward Laplace force and the outward elastic energy. Step 3: large unstable nanoparticles undergo digestive ripening or coalescence breakage, with increased PDI due to smaller particles. Step 4: size-focused region of stabilized quasi-monodispersed DBCO-SIRF-B1 nanoparticles controlled by the ACN concentration.

FIG. 3 are atomic force microscopy (AFM) and transmission electron microscopy (TEM) observations depicting the difference in surface profiles of DBCO-SIRF-B1 nanoparticles in the state of total coalescence (FIG. 3A) and binary arrested coalescence (FIG. 3B). FIG. 3C is a graph of the surface profile of a total coalescence particle showing that the interface between two independent particles is sharp as depicted by the black arrow, whereas arrested coalescence shows a smooth transition between the two conjoining particles. FIG. 3D is a series of AFM and TEM images to depict the different states of arrested coalescence from doublets to quadruplets. FIG. 3E is an AFM phase-contrast image of multiple self-assembled globules. FIG. 3F shows the zoom-in of a single 200 nm nanoparticle showing each small globules approximately 20 nm in diameter. FIG. 3G is a TEM image of a 150 nm nanoparticle showing areas of different globules' contrast. FIG. 3H is a digitally enhanced image of FIG. 3G. Surface blurring, noise removal and HDR toning was applied to improve the contrast and clarity of the internal globular elements.

FIG. 4 shows the structural colouration and reflectance of DBCO-SIRF-B1 nanoparticles monolayer film using Langmuir-Schaefer deposition method. Structural colouration of nanoparticles with average sizes of A) 170 nm, B) 240 nm, C) 270 nm and D) 310 nm was observed under light microscopy, together with its AFM image (middle) and reflectance measurements (bottom).

FIG. 5 shows the structural colouration and reflectance of DBCO-SIRF-B1 nanoparticles monolayer film using the dropcast deposition method. Average nanoparticle size of A) 400 nm, B) 460 nm, C) 520 nm and D) 660 nm dropcasted onto azide-functionalised wafers are shown with corresponding AFM topology images (second from top) of monolayer and reflectance measurements (third from top). White patches and lines are scratch marks on the soft nanoparticle coating arising from repeated handling. E)-F) show hydration-induced spectral shift of 460 nm DBCO-SIRFB1 nanoparticles monolayer film. In the dehydrated state E), the monolayer reflectance colour is blue, whereas in the state F), the reflectance colour is orange-red. FIG. 5G shows reflectance measurements showing the hydration-induced spectral red-shift with peak reflectance at 700 nm. The shift is reversible and can be obtained multiple times without any change in the reflectance spectra.

FIG. 6 shows the effect of coumarin 343× azide and 5-FAM azide having an absorbance at 430-500 nm on the 660 nm nanoparticle coatings, wherein FIG. 6A shows a 660 nm nanoparticle coating with blue structural coloration at 429 nm and an attenuated red peak at ca. 700 nm wavelength. FIG. 6B shows a 660 nm nanoparticle coating conjugated with coumarin 343× azide for 1 h exhibiting purple color due to the mixture of red and blue wavelengths. FIG. 6C shows that a conjugation time extended to 24 h greatly suppressed the secondary resonance peak at 429 nm, making the red wavelength dominant.

FIG. 7 shows the liquid-liquid phase separation of SIRF-B1 protein in the presence of a chaotropic agent, wherein the concentration is below the critical concentration required to completely solubilise SIRF-B1. Coacervate microdroplets of SIRF-B1 can be seen under transmitted light microscope with a diameter of 1-3 μm. The inset shows the dense viscous protein phase (orange colour) at the bottom of the centrifuge tube after two days which can only be re-solubilised by heating above 80° C.

FIG. 8 shows the DLS intensity size distribution of DBCO-SIRF-B1 nanoparticles self-assembled from 10 mM (3-(N-morpholino) propanesulfonic acid) (MOPS) buffered to pH 7.0 with 20-35% v/v acetonitrile and 5 mM DBCO-Sulfo-NHS ester.

FIG. 9 shows the growth of 360 nm nanoparticle and its respective PDI. This pattern is not stochastic and shows similarity to the 660 nm particle growth where the maximum average size corresponds to a decrease in PDI. Data was extrapolated after 800 s.

FIG. 10 is a TEM image of long chain coalescence necking between multiple particles observed when the coalescence process is halted prematurely after ten minutes. The final average particle size of this specific sample was 280 nm.

FIG. 11 is a schematic diagram of Langmuir-Blodgett and Langmuir-Schaefer deposition method using sodium polytungstate as the carrier medium. Langmuir-Blodgett deposition could not form a monolayer due to the thin layer of sodium polytungstate between the sample and wafer surface. A custom designed Langmuir-Blodgett/Schaefer mini device was used to prepare the Langmuir film on wafer. Langmuir-Schaefer deposition method proved to be more suitable for the fabrication of DBCOSIRF-B1 monolayer PASs.

FIG. 12 illustrates the surface functionalization of a wafer verified with contact angle measurements and ellipsometry. The thickness of each molecule layer was derived by subtracting the total thickness with the previous layer thickness. A) Piranha etch wafer was used as the negative control. B) With N-[3-(Trimethoxysilyl) propyl] ethylenediamine (AEAPTMS). C) With Azido-dPEG®4-TFP. D) Fluorescent 6-FAM DBCO used as the positive control to verify the presence of an azide on the surface. E) Non-functionalised wafer did not fluoresce under 488 nm excitation wavelength. F) Green fluorescence was observed for wafer functionalised with 6-FAM-DBCO.

FIG. 13 is an AFM of DBCO-SIRF-B1 nanoparticles. A) 215 nm, B) 270 nm, and C) 320 nm, and D) 375 nm average particle size with their respective fast Fourier transform (FFT) image (bottom row), indicating that all monolayers are arranged as photonic amorphous structures.

FIG. 14 shows the thermal-assisted colloidal self-assembly of 215 nm DBCO-SIRF-B1 nanoparticles azide-functionalised 100 mm² wafer at different temperatures. A) At room temperature the nanoparticles are concentrated near the centre of the wafer with a blue halo ring surrounding the sample. B) The coffee-ring effect of the nanoparticle at 40° C. is seen as concentric rings formed during the evaporation of the buffer. Structural colouration was present. C) The self-assembly of the nanoparticles at 50° C. shows attenuated structural colouration with a light blue hue on the low half of the sample. D) No structural colouration was observed for the sample heated to 60° C. The coating had surface unevenness with structural defects.

FIG. 15 shows AFM images of DBCO-conjugated SIRF-B1 nanoparticles of various diameter drop-casted onto azide-functionalised 225 mm² wafer at 40° C. and 23° C. At higher temperature, large empty patches are likely due to the rapid evaporation of acetonitrile forming pockets of convection currents, which prevent even distribution of nanoparticles. Samples prepared at room temperature show improved dispersion and smaller inter-particle spacing. Due to the high concentration and irregular arrangement of the particles, no structural colouration was observed in any of the samples under direct lighting.

FIG. 16 is a light microscopy image of A) 400 nm and B) 460 nm DBCO-SIRF-B1 monolayer PASs using drop casting deposition method to produce structural colouration on silicon wafer. Respective AFM image of region α) shows the topology of the monolayer film with small inter-particle spacing less than 1 μm where structural colouration was observed, and region B) with no structural colouration shows large inter-particle spacing.

FIG. 17 shows a UV-Vis spectrum of 400 nm and 600 nm DBCO conjugated and unconjugated SIRF-B1 nanoparticles in solution. The peak at 310 nm correspond to the presence of DBCO. The 200 nm and 280 nm peak for the unconjugated SIRF-B1 nanoparticle correspond to the peptide bond and aromatic amino acid signature. The DBCO conjugated nanoparticles show a decreasing absorbance observed in the visible wavelength.

FIG. 18 shows the custom-made setup to utilise track etched membranes for desalting of large volumes. Pore size of 100 to 800 nm was able to improve polydispersity index by filtering out small nanoparticles.

FIG. 19 shows a custom-made wafer support platform to functionalize multiple 100 mm² or 225 mm² wafers simultaneously.

FIG. 20 is a reaction scheme for functionalization of silicon dioxide surface and conjugation of SIRFB1-DBCO nanoparticle through copper-free click chemistry. A) Piranha etched silicon dioxide surface with generated hydroxyl groups and functionalised with [3-(2-Aminoethylamino) propyl] trimethoxysilane at 130° C. for 16 h. B) Azido-dPEG®4-TFP ester has specificity for the free amine group and left to react for at least 4 h. C) The free amine group is further reacted with Azido-dPEG®4-TFP ester to generate click chemistry ready azide group. D) Introduction of SIRF-B1-DBCO nanoparticle to the surface and copper-free click chemistry reaction left to proceed for 24 h. E) The ‘clicked’ DBCO-SIRF-B1 nanoparticle forms a covalent 1,4-disubstituted triazole with the azide-functionalised silicon dioxide surface.

FIG. 21 is a comparison of non-functionalised (left) and functionalised coverslip (right) with APTES, 4-ethynylbenzoic acid or propiolic acid, and 1-Azidomethylpyrene as the fluorescent probe excited under UV light to confirm successful functionalization.

FIG. 22 shows a reaction scheme of triethoxy (ethynyl) silane to hydroxylated glass surface after piranha etching and fluorescent probing with 1-Azidomethylpyrene.

FIG. 23 is an example reaction scheme of protein nanoparticle with surface populated with either carboxylic acid or amines that may be conjugated with azidoproylamine or azidoacetic acid through the use of EDC/NHS or DIC/HOBt chemistries.

FIG. 24A simple self-contained Langmuir-Blodgett and Langmuir-Schaefer setup using a small glass petri dish or a 6-well polystyrene plate. Sodium polytungstate density is adjusted accordingly based on the density of the sample and type of O-ring used. Left image, a schematic diagram of the setup with the masked substrate along the edge. Right image, an actual setup using a 6-well polystyrene plate. SIRF-B1 nanoparticles were contained within the FKM O-ring, giving off a faint purple hue (only in this example) after the particles are reasonably compacted as a monolayer.

FIG. 25 shows the wavelengths of UV-A, -B and -C.

FIG. 26 shows the absorbance of bulk titanium dioxide and zinc oxide at room temperature.

FIG. 27 shows the particle size dependence of TiO₂ on UV-A and UV-B properties.

FIG. 28 shows the UV-Vis absorbance spectra for the TiO₂ controls and commercial sunscreen.

FIG. 29 shows the UV absorbance of DBCO conjugated reflectin nanoparticles (400 nm) in solution. Concentration 0.3 mg mL⁻¹ diluted 250× (1.2 μg mL⁻¹). The UV profile of DBCO-sulfo-NHS ester and native SIRF-B1 was used as the control.

FIG. 30 is a 1-day cytocompatibility test of DBCO-SIRF-B1 (400 nm) and ZnO (250 nm) nanoparticles at concentrations of 2, 20 and 200 μL. DBCO-SIRF-B1 shows better keratinocyte tolerance at higher concentrations.

FIG. 31 is an Alamar blue staining of the cell control and incubation with different concentration of reflectin and ZnO nanoparticles.

FIG. 32 shows Keratinocyte cell uptake of 400 nm DBCO-SIRF-B1 nanoparticles confirmed with 5-FAM azide staining.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the disclosure. Other embodiments may be utilized and structural, and logical changes may be made without departing from the scope of the disclosure. The various embodiments are not necessarily mutually exclusive, as some embodiments can be combined with one or more other embodiments to form new embodiments.

In one aspect, the present disclosure provides a recombinant reflectin nanoparticle. Advantageously, by controlling an average size of the reflectin nanoparticles, the colour of films/coatings made of recombinant reflectin nanoparticles could be regulated. Accordingly, a biomimetic approach towards colour modulation is realized by the provision of a recombinant reflectin nanoparticle. Moreover, due to the recombinant reflectin nanoparticle being substantially monodisperse and/or with controllable size, an iridophores' photonic response could be mimicked. For example, it is possible to provide the recombinant reflectin nanoparticle with tunable size in the range of about 100 to about 1000 nm. By immobilizing the recombinant reflectin nanoparticle on a surface, it is possible to provide monolayer photonic structures with tunable structural colours, thereby allowing for the fabrication of eco-friendly, bioinspired colour-changing coatings that mimic the dynamic camouflage of cephalopods.

The recombinant reflectin nanoparticle may comprise a polypeptide resembling a naturally-occuring polypeptide sourced from a cephalopod. Said naturally-occuring polypeptide may have been fully sequenced and subsequently recombinantly expressed by bacteria, e.g., E. coli, before self-assembly. The naturally-occuring polypeptide sourced from a cephalopod may comprise a polypeptide called “reflectin”, typically made up of conserved amino acid sequences. Each sequence may include a combination of standard and sulphur-containing amino acids. Light interacting properties of the reflectin polypeptide may be attributed to its ordered hierarchical structure and hydrogen bonding.

In one example, the reflectin polypeptide is fully sequenced and recombinantly expressed. The fully sequenced reflectin polypeptide may be obtained from any family member of cephalopods. In one example, the reflectin polypeptide is obtained from Sepioteuthis lessioniana, and the reflectin polypeptide may be called reflectin B1. The sequence of Sepioteuthis lessoniana reflectin B1 is identified in Table 1 as SEQ ID NO: 1.

The reflectin polypeptide of reflectin B1 (SEQ ID NO:1) for use in the present disclosure may be any reflectin family or homolog thereof that substantially retains the activity of reflectin B1 (SEQ ID NO:1).

According to another aspect, there is thus provided a reflectin polypeptide comprising or consisting of:
(a) the amino acid sequence set forth in SEQ ID NO:1;
(b) an amino acid sequence that shares at least 65%, preferably at least 70%, or 75%, even more preferably at least 85%, most preferably at least 95% sequence identity with, or at least 80%, preferably at least 90%, more preferably at least 95% sequence homology with the amino acid sequence as set forth in SEQ ID NO:1;
(c) a functional fragment of (a) or (b); or
(d) an amino acid sequence containing either (a) or (b) or (c) as its essential component, wherein said reflectin polypeptide substantially retains the activity of reflectin B1 (SEQ ID NO: 1). In some embodiments, amino acid sequences pertaining to repeated motifs of Reflectin A1 are specifically excluded.

According to various embodiments, the reflectin polypeptide may be referred to as “substantially retaining” the activity of reflectin B1 (SEQ ID NO:1), when monolayer structures of the recombinant reflectin nanoparticle on a surface exhibit 80% of the structural colouration activity that is shown herein, at a temperature at or below 40° C.

According to various embodiments, the reflectin polypeptide comprises or consists of an amino acid sequence that is at least 60%, 65%, 70%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.25%, or 99.5% identical or homologous to the amino acid sequence set forth in SEQ ID NO: 1 over its entire length. In some embodiments, it has an amino acid sequence that shares at least 60, or at least 65, preferably at least 70, or at least 75, more preferably at least 80, most preferably at least 90% sequence identity with the amino acid sequence set forth in SEQ ID NO: 1 over its entire length or has an amino acid sequence that shares at least 80, preferably at least 90, more preferably at least 95% sequence homology with the amino acid sequence set forth in SEQ ID NO: 1 over its entire length.

The identity of nucleic acid or amino acid sequences is generally determined by means of a sequence comparison. This sequence comparison is based on the BLAST algorithm that is established in the existing art and commonly used, and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, which is calculated in relation to a reference sequence and its entire length. The term “sequence identity” refers to the extent that sequences are identical on a nucleotide-by-nucleotide or amino acid-by-amino acid basis over a window of comparison. Thus, a “percentage of sequence identity” is calculated by comparing two optimally aligned sequences over the window of comparison, determining the number of positions at which the identical nucleic acid base or amino acid residue occurs in both sequences to yield the number of matched positions, dividing the number of matched positions by the total number of positions in the window of comparison (i.e., the window size), and multiplying the result by 100 to yield the percentage of sequence identity. The more broadly construed term “homology”, in the context of amino acid sequences, also incorporates consideration of the conserved amino acid exchanges, i.e. amino acids having a similar chemical activity, since these usually perform similar chemical activities within the protein. The similarity of the compared sequences can therefore also be indicated as a “percentage homology” or “percentage similarity.” Indications of identity and/or homology can be encountered over entire polypeptides or genes, or only over individual regions. Homologous and identical regions of various nucleic acid sequences or amino acid sequences are therefore defined by way of matches in the sequences. Such regions often exhibit identical functions. They can be small, and can encompass only a few nucleotides or amino acids. Small regions of this kind often perform functions that are essential to the overall activity of the protein. It may therefore be useful to refer sequence matches only to individual, and optionally small, regions. Unless otherwise indicated, however, indications of identity and homology herein refer to the full length of the respectively indicated nucleic acid sequence or amino acid sequence.

All amino acid residues are generally referred to herein by reference to their one letter code and, in some instances, their three letter code. This nomenclature is well known to those skilled in the art and used herein as understood in the field.

The reflectin polypeptide substantially retaining the activity of reflectin B1 (SEQ ID NO: 1) according to the present application can comprise amino acid modifications, in particular amino acid substitutions, insertions, or deletions. Such reflectin polypeptides can be, for example, further developed by targeted genetic modification, i.e. by way of mutagenesis methods, and optimized for specific purposes or with regard to special properties (for example, with regard to their ability to form nanoparticles and/or providing for tunable structural colours, etc.). The objective may be to introduce targeted mutations, such as substitutions, insertions, or deletions, into the known molecules in order, for example, to improve their ability to form nanoparticles and exhibit structural colouration in the form of a monolayer. For this purpose, in particular, the surface charges and/or isoelectric point of the molecules, and thereby their interactions with the substrate, can be modified. Advantageous properties of individual mutations, e.g. individual substitutions, can supplement one another.

In various embodiments, the reflectin polypeptide may be characterized in that it is obtainable from a reflectin as described above as an initial molecule by single or multiple conservative amino acid substitution. The term “conservative amino acid substitution” means the exchange (substitution) of one amino acid residue for another amino acid residue, where such exchange does not lead to a change in the polarity or charge at the position of the exchanged amino acid, e.g. the exchange of a nonpolar amino acid residue for another nonpolar amino acid residue. Conservative amino acid substitutions in the context of the disclosure encompass, for example, G=A=S, I=V=L=M, D=E, N=Q, K=R, Y=F, and S=T.

The reflectin polypeptide may be a recombinant reflectin polypeptide, i.e. reflectin produced in a genetically engineered organism that does not naturally produce said reflectin polypeptide. The term “recombinantly express” as used herein refers to the expression of said reflectin polypeptide by recombinant DNA technology, using nucleic acid molecules. The nucleic acid molecules encoding the reflectin polypeptide described herein, as well as a vector containing such a nucleic acid, in particular a copying vector or an expression vector also form part of the present disclosure.

Accordingly, there is also provided for a nucleic acid molecule encoding the reflectin polypeptide as described herein. In some embodiments, the nucleic acid molecule may be comprised in a vector. The vector may further comprise regulatory elements for controlling expression of said nucleic acid molecule.

“Vectors” are understood for purposes herein as elements-made up of nucleic acids that contain a nucleic acid contemplated herein as a characterizing nucleic acid region. They enable said nucleic acid to be established as a stable genetic element in a species or a cell line over multiple generations or cell divisions. In particular when used in bacteria, vectors are special plasmids, i.e. circular genetic elements. In the context herein, a nucleic acid as contemplated herein is cloned into a vector. Included among the vectors are, for example, those whose origins are bacterial plasmids, or predominantly synthetic vectors or plasmids having elements of widely differing derivations. Using the further genetic elements present in each case, vectors are capable of establishing themselves as stable units in the relevant host cells over multiple generations. They can be present extrachromosomally as separate units, or can be integrated into a chromosome resp. into chromosomal DNA.

The Expression vectors may encompass nucleic acid sequences which are capable of replicating in the host cells, preferably bacteria, that contain them, and expressing therein a contained nucleic acid. In various embodiments, the vectors described herein thus also contain regulatory elements that control expression of the nucleic acids encoding the reflectin polypeptide as described herein. One example of such a vector may be a pET vector. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. Expression vectors can furthermore be regulated, for example by way of a change in culture conditions or when the host cells containing them reach a specific cell density, or by the addition of specific substances, in particular activators of gene expression. One example of such a substance is the galactose derivative isopropyl-beta-D-1-thiogalactopyranoside (IPTG), e.g., a T7 promoter.

In some embodiments, the isoelectric point of the reflectin polypeptide may be above 7, or above 8, or between about 8 and 10. Advantageously, at such high isoelectric points, there are opportunities provided for modulation of the zeta potential and thus colloidal characteristic by screening buffer type and additives during the purification (e.g., dialysis) process. For example, during a dialysis step, the following criteria could simultaneously be achieved: (i) mitigation of aggregation, (ii) control of nanoparticle size, (iii) narrow size distribution, and (iv) particle stability.

The reflectin polypeptide obtained from the expression may be self-assembled into a nanoparticle, thereby forming a recombinant reflectin nanoparticle. A “nanoparticle” refers to a particle having a characteristic length, such as diameter, in the range of below 1000 nm. The recombinant reflectin nanoparticle may have a regular shape, or may be irregularly shaped. For example, the recombinant reflectin nanoparticle may be a sphere, a rod, a cube, or irregularly shaped. The size of the recombinant reflectin nanoparticle may be characterized by its mean diameter. The term “diameter” as used herein refers to the maximal length of a straight line segment passing through the center of a figure and terminating at the periphery. The term “mean diameter” refers to an average diameter of the nanoparticle, and may be calculated by dividing the sum of the diameter of each nanoparticle by the total number of nanoparticles. Although the term “diameter” is used normally to refer to the maximal length of a line segment passing through the centre and connecting two points on the periphery of a nanosphere, it is also used herein to refer to the maximal length of a line segment passing through the centre and connecting two points on the periphery of nanoparticles having other shapes, such as a nanocube or a nanotetrahedra, or an irregular shape.

The self-assembly into the nanoparticle may be carried out either with an unconjugated reflectin polypeptide or with a reflectin polypeptide that is conjugated (e.g., ligated) to a ligand. For example, the reflectin polypeptide to be ligated in accordance with the present application may be modified by conjugation to a ligand, before or after self-assembly of the reflectin polypeptide into the nanoparticle. The unconjugated ligand (i.e., the chemical structure of the ligand before conjugation) may comprise a functional group for reaction with the reflectin polypeptide. More particularly, in various embodiments, the functional group of the unconjugated ligand may comprise a leaving group, commonly used for making a peptide bond. The functional group may be a succinimide ester, or a fluorinated phenyl ester. The succinimide of the succinimide ester or the fluorinated phenyl of the fluorinated phenyl ester may function as a leaving group in a reaction with a free amine of the reflectin polypeptide. Hence, in some embodiments, the conjugation of the reflectin polypeptide to the ligand may result in a covalent bond between the ligand and the reflectin polypeptide. The covalent bond between the reflectin polypeptide and the ligand may be a peptide bond, i.e. an amide bond.

In embodiments where the functional group of the unconjugated ligand for reaction with the reflectin polypeptide is a succinimide ester, the succinimide may be modified with an electron-withdrawing group. The electron-withdrawing group may comprise an SO₃⁻ group.

The ligand may further comprise a connecting group for immobilizing the recombinant reflectin nanoparticle to a substrate. Said connecting group may comprise, or be, a triple bond. The triple bond may react with an azide that may be linked to a substrate, in order to form a covalent bond between the substrate and the ligand. Alternatively, the connecting group may be an azide, while a triple bond may be linked to the surface that is to be functionalized for covalent bonding of the ligand to the surface. Hence, in some embodiments, the immobilisation of the recombinant reflectin nanoparticle to the azide via the ligand may result in a covalent bridge between the recombinant reflectin nanoparticle and the surface via the ligand.

In some embodiments, the ligand (after conjugation) may have the following formula (I):

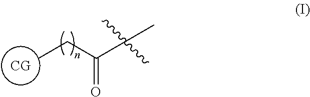

wherein CG is the connecting group, and n is an integer selected from 1 to 5, e.g., n may be 1, 2, 3, 4, or 5, and [symbol] indicates the point of connection to a nitrogen atom of the reflectin polypeptide for formation of the peptide bond.

The connecting group CG may be a moiety of the following formula (II):

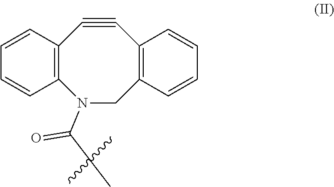 [SMILES: *C(=O)N1Cc2ccccc2C#Cc2ccccc21]

and [symbol] indicates the point of connection to the —(CH₂) n moiety in the ligand of Formula (I). Alternatively, the connecting group CG may be an azide (—N₃).

In one example, the unconjugated ligand may be dibenzocyclooctyne-sulfo-NHS ester (DBCO-Sulfo-NHS ester), or its sodium salt.

In various embodiments, the recombinant reflectin nanoparticles are controlled to essentially be of the same size, i.e., they may be substantially monodisperse. For measuring the heterogeneity of a sample based on size, the polydispersity index (PDI) is often used. Polydispersity can occur due to size distribution in a sample or agglomeration or aggregation of the sample during isolation or analysis. The PDI can be obtained from instruments that use dynamic light scattering (DLS) or determined from electron micrographs. The PDI for the recombinant reflectin nanoparticle may be below 0.5. Advantageously, for embodiments where the recombinant reflectin nanoparticle nanoparticle is conjugated to a ligand, the PDI may be below 0.1, or below 0.09.

In various embodiments, the recombinant reflectin nanoparticle may have a high negative zeta potential. The zeta potential may be understood as a measurable indicator of the stability of the colloidal dispersion, wherein the magnitude of the zeta potential may indicate the degree of electrostatic repulsion between adjacent, similarly charged particles in the dispersion. In other words, a high zeta potential may confer stability, i.e., the solution or dispersion will resist aggregation. In contrast, when the potential is small, attractive forces may exceed this repulsion and the dispersion may break and flocculate. So, colloids with high zeta potential (negative or positive) are electrically stabilized while colloids with low zeta potentials tend to coagulate or flocculate. According to various embodiments, the zeta potential of the recombinant reflectin nanoparticle may be in the range of about −30 to −100 mV, or in the range of −35 to −45 mV, indicating a stable colloidal suspension.

In various embodiments, the recombinant reflectin nanoparticle may be crystalline. Crystalline photonic structures may provide long-range order resulting in iridescence. In alternative embodiments, the recombinant reflectin nanoparticle may be amorphous. Amorphous photonic structures may provide short-range order resulting in structural colouration. In one example, the recombinant reflectin nanoparticle may have an amorphous photonic structure.

In a further aspect, the disclosure is also directed to a host cell, preferably a non-human host cell, containing a nucleic acid molecule as contemplated herein or a vector as contemplated herein. A nucleic acid as contemplated herein or a vector containing said nucleic acid is preferably transformed into a microorganism, which then represents a host cell according to an embodiment. Methods for the transformation of cells are established in the existing art and are sufficiently known to the skilled artisan. All cells are in principle suitable as host cells, i.e. prokaryotic or eukaryotic cells. Those host cells that can be manipulated in genetically advantageous fashion.

Preferred host cells are prokaryotic or bacterial cells, such as E. coli cells. Bacteria are notable for short generation times and few demands in terms of culturing conditions. As a result, economical culturing methods resp. manufacturing methods can be established. In addition, the skilled artisan has ample experience in the context of bacteria in fermentation technology. Gram-negative or Gram-positive bacteria may be suitable for a specific production instance, for a wide variety of reasons to be ascertained experimentally in the individual case, such as nutrient sources, product formation rate, time requirement, etc. In various embodiments, the host cell may be E. coli cells.

Host cells contemplated herein can be modified in terms of their requirements for culture conditions, can comprise other or additional selection markers, or can also express other or additional proteins.

The host cells contemplated herein are cultured and fermented in a usual manner, for example in discontinuous or continuous systems. In the former case a suitable nutrient medium is inoculated with the host cells, and the product is harvested from the medium after a period of time to be ascertained experimentally. Continuous fermentations are notable for the achievement of a flow equilibrium in which, over a comparatively long period of time, cells die off in part but are also in part renewed, and the protein formed can simultaneously be removed from the medium. Host cells contemplated herein are preferably used to manufacture the reflectin described herein.

A further aspect of the disclosure is therefore a method for synthesizing a reflectin polypeptide as described herein, comprising culturing a host cell contemplated herein; and isolating the reflectin polypeptide from the culture medium or from the host cell. Culture conditions and mediums can be selected by those skilled in the art based on the host organism used by resorting to general knowledge and techniques known in the art.

In a further aspect, there may be provided a method of synthesizing a recombinant reflectin nanoparticle. The method may comprise a first step of recombinantly expressing a reflectin polypeptide as described herein. This expression may be carried out from E. coli. In other words, the method may comprise the steps of providing an E. coli host cell culture, changing the growth rate of the E. coli host cells and inducing expression of the recombinant reflectin polypeptide, as inclusion bodies. Recombinantly expressed inclusion bodies may then be extracted from the E. coli host cell culture. In a next step, these inclusion bodies may be solubilized. The solubilizing may be carried out under strong denaturing conditions, for example, using a urea solution or a dimethylurea solution having a molar concentration of about 5 to 10. Having a molar concentration in this range may be advantageous for transparency of the recombinant reflectin solution. Additionally or alternatively, the solution may be heated to a temperature of about or above 80° C., or between 80° C. to about 100° C.

After the recombinant reflectin polypeptide is solubilized, the next step may be a purification by chromatography. Advantageously, the purification by chromatography may be carried out at a pH range higher than pH 5.0, which may advantageously be beneficial for the stability of the recombinant reflectin polypeptide. In various embodiments, the chromatography may be carried out using ion-exchange chromatography, e.g., cation-exchange chromatography. After purification, a purity of higher than 95%, or higher than 98% of the recombinant reflectin polypeptide may be obtained.

In some embodiments, the recombinant reflectin polypeptide may be conjugated to the ligand described herein before, which may be carried out before self-assembly. The conjugation of the ligand may involve adding the unconjugated ligand in a solution. The unconjugated ligand may have a molar concentration in the solution of about 5 mM to 10 mM. Below this molar concentration range, the solution may not be stabilized, while a molar concentration above this range may not further affect the size or stability of the ensuing recombinant reflectin nanoparticles.

After chromatography of the recombinant reflectin polypeptide and optional conjugation to a ligand, a dialysis step may follow for the removal of the urea or dimethylurea. The dialysis step may advantageously include the self-assembly step.

Thus, in a next step, the purified recombinant reflectin polypeptide may be triggered to undergo self-assembling into a nanoparticle. This step may be effected in pure water. Alternatively, in some embodiments, a buffer solution may be added. The buffer solution may have a molar concentration of about 5 to about 20 mM. The type of buffer may be a Good's buffering agent, or be selected from the group consisting of MOPS, MES, HEPES, and a combination thereof. The pH of the buffer solution may be modulated between 4 and 10, or between 6 and 8, and in some embodiments, about 7.0 to 7.4.

The buffer solution may comprise an organic solvent. The organic solvent may be a polar solvent. More particularly, the organic solvent may be selected from the group consisting of a polar aprotic solvent or an alcohol. The polar protic solvent may be selected from the group consisting of acetone, acetonitrile, dimethylformamide, dimethylpropyleneurea, dimethylsulfoxide, hexamethylphosphoric triamide, pyridine, sulfolane, tetrahydrofuran, and a combination thereof. The alcohol may be selected from the group consisting of methanol, ethanol, iso-propanol, tert-butanol and a combination thereof.

The concentration of the organic solvent in the buffer solution may be about 5 to 50%, or about 20 to 35%, or about 5 to 15%. Advantageously, the size of the recombinant reflectin nanoparticle may be controlled with the concentration of the organic solvent. For example, when using acetonitrile as the organic solvent, it may be possible to obtain a substantially linear relationship of the size of the nanoparticle with increasing concentration of the acetonitrile between 10 to 20%, and 20 to 30%.

When no organic solvent is added for the self-assembly, the pH of the aqueous buffer may be about 8 to 10, e.g., by using Good's buffering agent, or by using 5 to 50 mM of sodium borate or 50 to 200 mM of imidazole. Optionally, sodium chloride may be added.

In some embodiments, surfactants may be added to the solution in which the self-assembly is to be carried out. The surfactant may either be zwitterionic or neutral.

In some embodiments, anti-oxidants may be added to the solution in which the self-assembly is to be carried out. The anti-oxidant may be ascorbic acid and/or sodium ascorbate.

In some embodiments, methyl-β-cyclodextrin may be added to the solution in which the self-assembly is to be carried out. Advantageously, by adding methyl-β-cyclodextrin, the size of the ensuing recombinant reflectin nanoparticle may be controlled to be below 200 nm.

In another aspect, there is provided a substrate surface-functionalized with a recombinant reflectin nanoparticle. Advantageously, by using the recombinant reflectin nanoparticle with a specific nanoparticle size, it is possible to trigger reflectance exhibiting a tunable response from violet (400 nm) to near infrared-red (800 nm). The recombinant reflectin nanoparticle immobilized on the substrate also allows for dynamic colour-changing, triggered by hydration-induced swelling of the recombinant reflectin nanoparticle.

In some embodiments, the recombinant reflectin nanoparticle may be assembled on the surface substantially as a monolayer. In some embodiments, the recombinant reflectin nanoparticle may be covalently immobilized on the surface, optionally using a drop-cast deposition method. In some embodiments, the distance of one recombinant reflectin nanoparticle to another recombinant reflectin nanoparticle is less than 1 micrometer.

In another aspect, there is provided a method of immobilizing a recombinant reflectin nanoparticle on a substrate, the method comprising providing a substrate comprising hydroxy groups; reacting the hydroxy groups with a surface-bound spacer chain; providing a recombinant reflectin nanoparticle and reacting the recombinant reflectin nanoparticle with the surface-bound spacer chain.

The substrate may comprise any material provided that it provides hydroxy groups on its surface for surface-treatment, e.g., glass. These hydroxy groups may be surface-treated with an organosilane, which may be one example of a surface-bound spacer chain. Advantageously, by surface-treating the substrate with an organosilane, hydrogen bonding and/or a covalent bond between the substrate and the recombinant reflectin nanoparticle may be facilitated. Surface treatment with an organosilane may be used to covalently bond the surface-treated substrate with the recombinant reflectin nanoparticle. In the event the organosilane is present and linked to the recombinant reflectin nanoparticle, a “covalent bridge” may be formed stretching from the substrate via the surface-bound spacer chain to the recombinant reflectin nanoparticle.

The organosilane may comprise an active functional group selected from the group consisting of octyl, amine, vinyl, ethynyl, hydroxyl, thiol, and a combination thereof. The organosilane may be an aminoalkylsilane, e.g., APTES, or triethoxy (ethynyl) silane. In some embodiments, the organosilane may be further functionalized with a further linker, such that a click chemistry functional group (i.e., an azide or a triple bond) is at a terminal position to enable covalent bond formation via click-chemistry with the recombinant reflectin nanoparticle, e.g., via the connecting group of the ligand. Accordingly, both the surface-functionalized substrate and the recombinant reflectin nanoparticle, before reaction with each other, may have a complementary click-chemistry functional group. In other words, one of the components may have a triple bond functionality, while the other has an azide functionality.

In another aspect, there is provided a skincare product comprising a recombinant reflectin nanoparticle. Advantageously, the absorption capacity of the recombinant reflectin nanoparticle may be utilized for a skincare product, such as sunscreen. More advantageously, it was found that the recombinant reflectin nanoparticle has a lower toxicity than conventionally used components in sunscreens, such as titanium oxide. Accordingly, in another aspect, there is further provided a skincare product for use in therapy. In particular, there is provided a skincare product for use in prevention of skin cancer and/or an inflammatory reaction to ultraviolet (UV) radiation damage to the skin's outermost layers (e.g., sunburn). There is also provided use of a skincare product in the manufacture of a medicament for the prevention of skin care and/or an inflammatory reaction to ultraviolet (UV) radiation damage to the skin's outermost layers.

The size of the nanoparticle that may be beneficial for use as a skincare product may be about 350 nm to about 450 nm, or about 400 nm.

The term “comprising” shall be understood to have a broad meaning similar to the term “including” and will be understood to imply the inclusion of a stated integer or operation or group of integers or operations but not the exclusion of any other integer or operation or group of integers or operations. This definition also applies to variations on the term “comprising” such as “comprise” and “comprises”.

By “about” in relation to a given numerical value, such as for concentration and composition, it is meant to include numerical values within 10% of the specified value.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

As used herein, the term “and/or” includes any and all combinations of one or more of the associated listed items.

### EXAMPLES

Reflectins from Sepioteuthis lessioniana squid were sequenced and the sequenced reflectin B1 was used to prepare coatings with tunable structural colouration. Reflectin B1 was conjugated to a click-chemistry ligand and self-assembled into quasi-monodispersed nanoparticles with tunable size in the 100 to 1000 nanometers (nm) range. Using Langmuir Schaefer and drop-cast deposition methods, ligand-conjugated reflectin B1 nanoparticles were immobilised onto azide-functionalised substrates via click-chemistry to produce monolayer amorphous photonic structures with tunable structural colours, paving the way for the fabrication of eco-friendly, bioinspired colour-changing coatings that mimic the dynamic camouflage of cephalopods. In one embodiment of this disclosure, a rechargeable energy storage system is based on the utilization and functionalization of safe, green, and sustainable rainwater as electrolyte.

Since the size of the reflectin nanoparticles govern the iridescence properties, the colour of films/coatings made of recombinant reflectin nanoparticles could be regulated by controlling their average size, followed by immobilizing them into photonic structures, mimicking in vivo ID Bragg lamellae photonic lattices. To this end, reflectin was first sequenced from S. lessoniana by next-generation RNA-sequencing (RNA-seq) of the dermal tissue (for clarity, unconjugated S. lessoniana B1 reflectin was termed as SIRF-B1). Full-length SIRF-B1 was then recombinantly expressed in E. coli and purified by a one-step strong cationic exchange chromatography. A systematic approach followed to self-assemble SIRF-B1 into discrete nanoparticle sizes exhibiting a polydispersity index (PDI) of less than 0.1 when conjugated with the click chemistry ligand dibenzocyclooctyne-sulfo-NHS ester (DBCO-Sulfo-NHS ester), which was achieved by varying the solvent conditions during the self-assembly process.

These quasi-monodispersed DBCO-SIRF-B1 nanoparticles were immobilised onto azide-functionalised wafer substrates by combining click chemistry with Langmuir-Schaefer and drop-cast deposition method, resulting in monolayer assemblies behaving as photonic amorphous structures (random close-packed particles), with reflectance exhibiting tunable response from violet (400 nm) to near infrared-red (800 nm) that were controlled by the nanoparticle size. Dynamic colour-changing of the monolayer film was also demonstrated, triggered by hydration-induced swelling of immobilised DBCO-SIRF-B1 nanoparticles. The formation and self-assembly of reflectin B1 nanoparticles with low polydispersity index and highly controlled tunable diameters rely solely on dialysis buffers at room temperature (23° C., 60% humidity) and atmospheric pressure without the use of any equipment. All of the chemicals used are of low toxicity and biocompatible.

### Advantages and Improvements Over Existing Methods, Devices or Materials

Self-assembly of proteins nanoparticle can be accomplished by supercritical fluid technology, emulsification, desolvation, complex coacervation, electrospray and sol-gel, but are challenging because of its susceptibility to chemical and physical degradation during processing, which involve stresses (heat, pressure, organic solvents) which is potentially detrimental to the protein's structure and function. A chemical method of producing protein nanoparticles involve co-lyophilising with methyl-β-cyclodextrin and resuspending in ethyl acetate. The control over nanoparticle size is limited and aggregation of the protein in the organic solvent is significant. This invention only involves mild and uncomplicated procedures for the self-assembly of protein nanoparticles which do not compromise protein integrity.

### Technical Description of the Invention

### Example 1: Expression, Purification, Self-Assembly and Conjugation

Recombinantly expressed SIRF-B1 (Table 1) may be characterized with the sequence below.

TABLE 1
Sepioteuthis lessoniana reflectin B1 sequence

MSSFMDPMHY DGMGMSHSKS GDFSHNCMRS FHKSQRDVMR
RDIMGKSNKN RRFGNLMEPM SRMTMDFHGR LIDSQGRIVD
PGHYFAMDDH YMENERFLYP HDMIRNRHGM YGFMQGDNVY
GNNMHRGMFA DGMYRDMHHS GMHPSGYMHG GSMQNRPMMY
MQGRYPDDSY FMNYHPSMSD PPVIMHSHYN DHEGRHGMYG
RPSESYGSHG RHGDAHSMPR RVSDTHSPQR RPSESHITQV
RPEGGSSRKT SRAKLFPDDK NTDSA

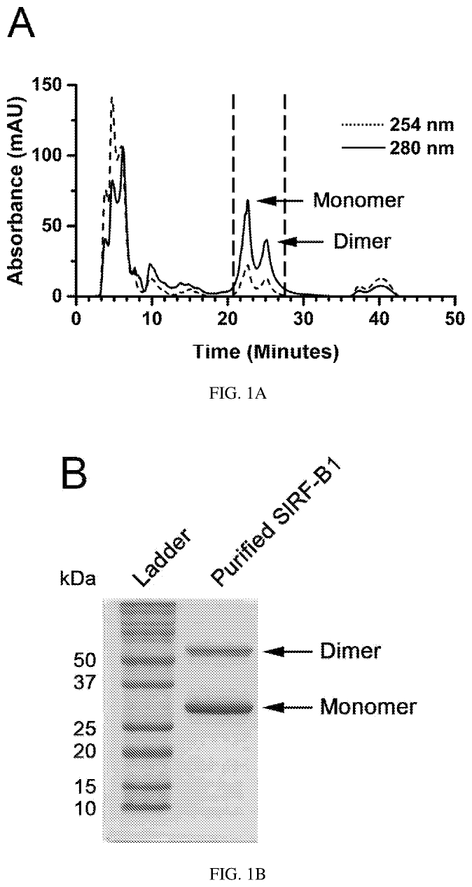

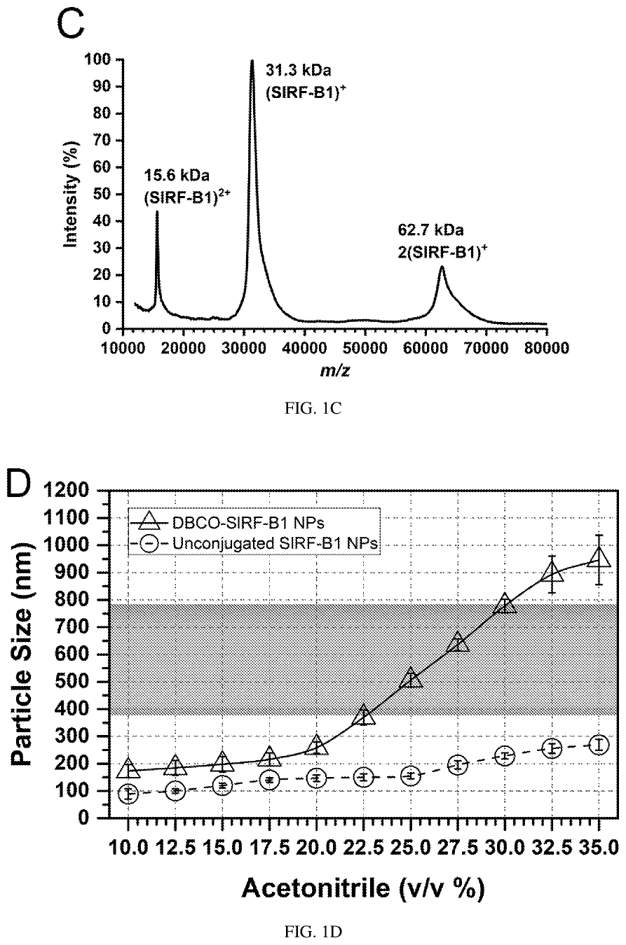

The recombinantly expressed SIRF-B1 inclusion bodies extracted from E. coli could be completely solubilised only under strong denaturing conditions (8 M urea or 6 M dimethylurea, T=85° C.). Below this chaotropic concentration, the initially transparent protein solution turned turbid and subsequently phase-separated through a liquid-liquid phase separation (LLPS) process, resulting in a condensed colloidal phase in a few days. The turbid solution was imaged by transmitted light microscopy and spherical coacervate microdroplets of SIRF-B1 with diameters in the 1-3 micrometer (μm) range were observed (FIG. 7). The settled protein phase was highly viscous and sticky (FIG. 7, inset) and could only be solubilised by heating the solution at 80° C. or higher. This process was reversible during subsequent cooling/heating cycles, indicating that SIRF-B1 exhibits an upper solution temperature (UPST) phase behaviour. The solubilised inclusion body was purified by a one-step strong cation-exchange chromatography, which yielded a purity estimated to be above 98% based on SDS-PAGE and MALDI-TOF analyses (FIG. 1A to FIG. 1C). The use of reversed phase high-pressure liquid chromatography (RP-HPLC) for purification was excluded as reflectin protein were found to degrade≤ pH 5.0.

In order to self-assemble SIRF-B1 into nanoparticles, dialysis protocols were used to gradually remove urea from the ion-exchange purified sample. One intrinsic property of SIRF-B1 is that it is a highly charged protein (isoelectric point of 8.8), providing opportunities to modulate its zeta potential and thus colloidal characteristic by screening buffer type and additives during the dialysis process. During dialysis, the following four criteria were thus simultaneously achieved: (i) mitigation of aggregation, (ii) control of nanoparticle size, (iii) narrow size distribution, and (iv) particle stability. To subsequently immobilise SIRF-B1 nanoparticle onto selected surfaces, click chemistry was used. Free amines on the surface of SIRF-B1 nanoparticles were first functionalised with different NHS ester-containing click chemistry molecules (see, Table 2), with copper-free click chemistry DBCO-Sulfo-NHS ester giving the best results in terms of size distribution.

TABLE 2
List of click chemistry molecules conjugated to SlRF-B1 nanoparticle and analysis of polydispersity after conjugation measured by
dynamic light scattering (DLS).
Selection of ligand for conjugation to self-assembled SlRF-B1 nanoparticle

| | | | | After | After | |
|---|---|---|---|---|---|---|
| Ligand | M.W. | Structure | Solubility | conjugation | desalting | DLS |
| Azidoacetic NHS Ester | 198.1 | 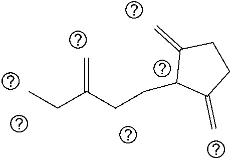 | Water, DMSO | Clear | Clear | Highly polydispersed |
| Azidobutyric NHS Ester | 226.2 | 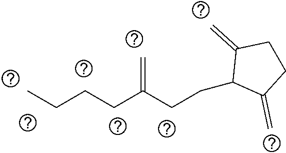 | Water, DMSO | Clear | Clear | Highly Polydispersed |
| Azido-PEG₃- NHS Ester | 344.3 | 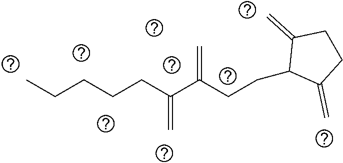 [SMILES: C=C(CCCCC)C(=C)CCC1C(=C)CCC1=C] | Water | Minor Aggregate | Aggregate | — |
| Azido-PEG₄- NHS Ester | 388.4 | 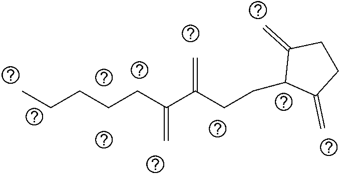 [SMILES: C=C(CCCCC)C(=C)CCC1C(=C)CCC1=C] | Water | Clear | Aggregate | — |
| Azido- dPEG ®₄-TFP | 439.4 | 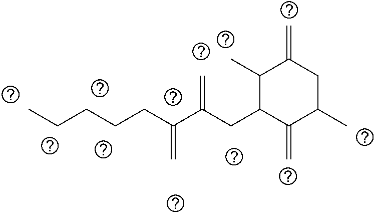 [SMILES: C=C(CCCCC)C(=C)CC1C(=C)C(C)CC(=C)C1C] | DMSO, DMF | Aggregate | — | — |
| DBCO-NHS Ester | 402.4 | 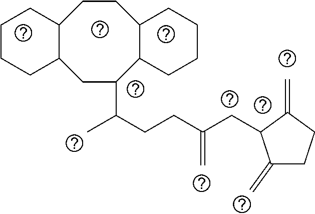 [SMILES: C=C(CCC(C)C1CC2CCCCC2CCC2CCCCC21)CC1C(=C)CCC1=C] | DMSO | Minor Aggregate | Aggregate | — |
| DBCO-Sulfo- NHS Ester | 532.5 | 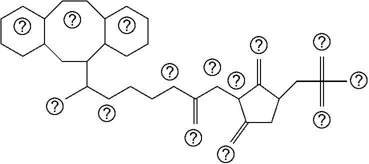 | Water, DMSO | Clear | Clear | Excellent PDI |

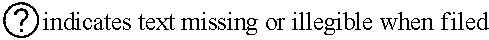

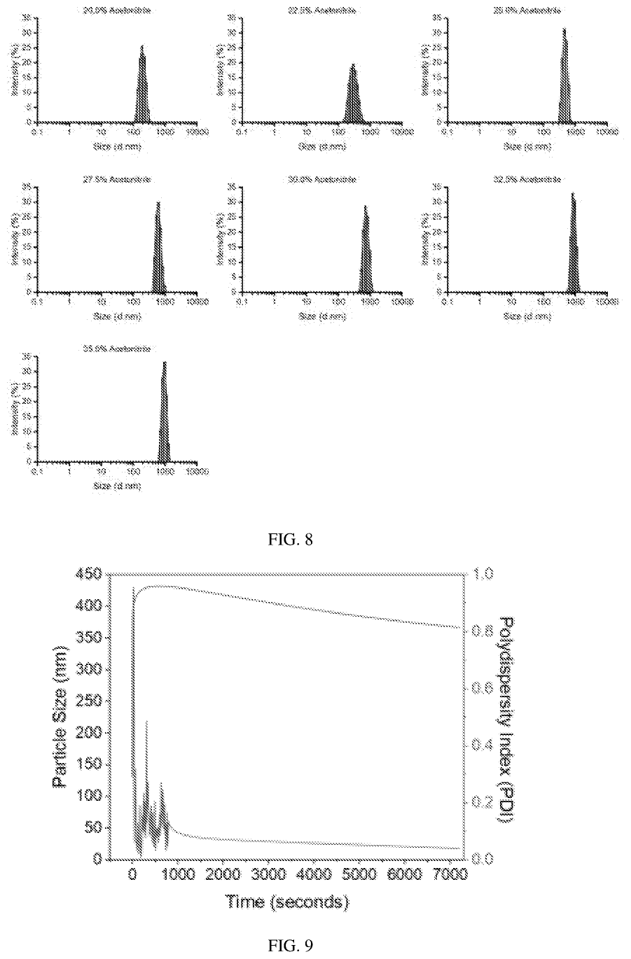

To control particle size, the acetonitrile (ACN) concentration was varied during dialysis as a way to modulate hydrophobic interactions between SIRF-B1 and solvent (FIG. 8). Polydispersity index (PDI) measured by Dynamic Light Scattering (DLS) improved from 0.40±0.20 (unconjugated) to less than 0.08±0.02 (conjugated) for most samples.

As shown in FIG. 1D, the final average size of the conjugated DBCO-SIRF-B1 nanoparticles could be precisely tuned between 200 nm and 1000 nm by increasing the ACN concentration from 20-35% v/v, with an almost linear correlation between particle size and ACN content. The ζ-potential for all samples was consistently ζ=−39.4±1.95 mV, indicating stable colloidal suspensions. The conjugation reaction was completed in as little as thirty minutes for particles ≤300 nm and in two hours for larger nanoparticles.

### Example 2: Growth Mechanism of DBCO-SIRF-B1 Nanoparticles

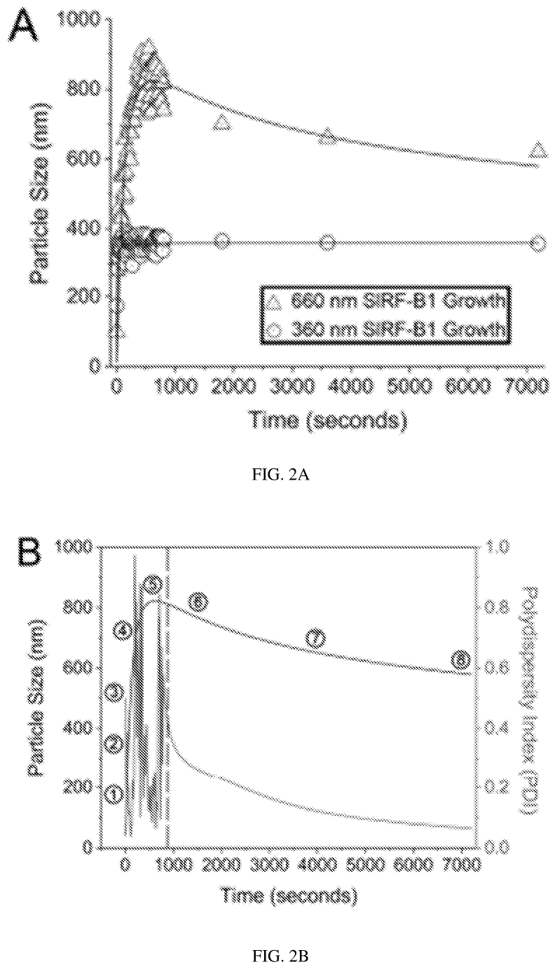

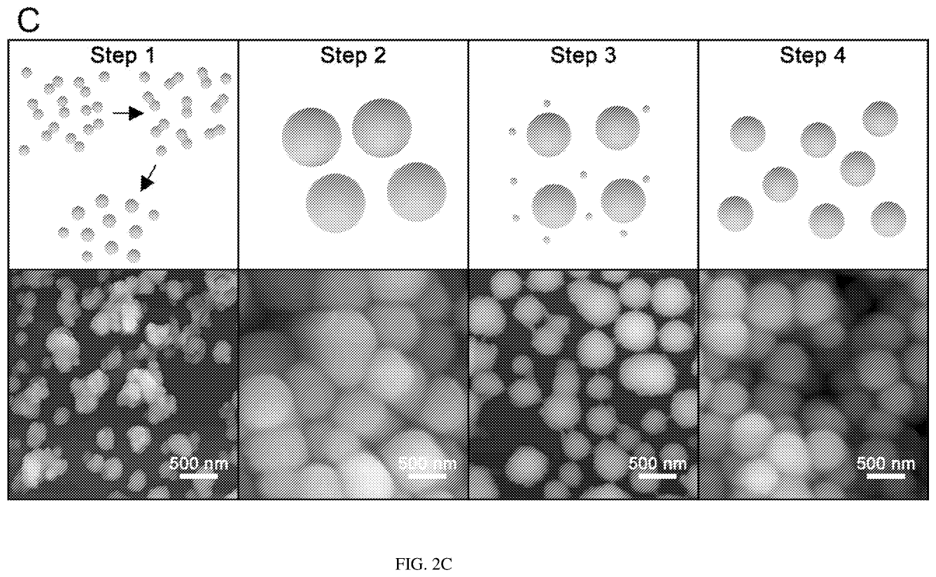

In determining which growth mechanism-LaMϵr burst nucleation, Ostwald/digestive ripening or coalescence—best describes the formation of DBCO-SIRF-B1 nanoparticles, the growth of 360 nm and 660 nm nanoparticles using DLS was monitored. A scatter plot of the particle size as a function of time is shown in FIG. 2A. The growth of the 360 nm nanoparticles was completed within a few minutes, with the final size obtained after 1000 s without further change over time. No additional peaks of small particle sizes that would be consistent with an Ostwald ripening mechanism were detected. For the 660 nm nanoparticles, an initial steep growth with a maximum average particle size of ca. 900 nm after 500 s was observed. The particle size then gradually decreased and reached its saturation plateau with a final average size of 660 nm. The data suggest that the growth initiation by DBCO is rapid, but formation of monodispersed particles requires more time for larger particle sizes. The growth curve of the DBCO-SIRF-B1 660 nm nanoparticles was further supplemented with the PDI at respective time points (FIG. 2B). Interestingly an initial rise was observed, and then fall in PDI. This pattern was not stochastic and was also observed for the 360 nm nanoparticles (FIG. 9).

The growth of DBCO-SIRF-B1 nanoparticles is suggested to occur primarily by a simplified 4-step coalescence mechanism, with a potential minor contribution of digestive ripening in the later stages, as schematically described in FIG. 2C. For clarity of the schematic model, only binary coalescence (merger of two particles) is illustrated. The self-assembled SIRF-B1 suspension was initially transparent, with growth initiated upon addition of DBCO-Sulfo-NHS ester. In step 1, the growth was rapid and exponential, forming a turbid yet opalescent yellow-orange solution. The large oscillating PDI could be explained by the anisotropic particle shape during coalescence. In step 2, the maximum particle size was reached when an equilibrium between the inward Laplace force and the outward elastic energy was reached; thus particles in this region have a low PDI. In step 3, the large unstable particles underwent digestive ripening, coalescence breakage, collisional breakage, whereby large particles shed excess protein material. Smaller particles once again grew through coalescence with decreasing PDI. Finally, step 4 is the final plateau region, where DBCO-SIRF-B1 stabilized into the final nanoparticle size with a narrow size distribution regulated by the ACN concentration.

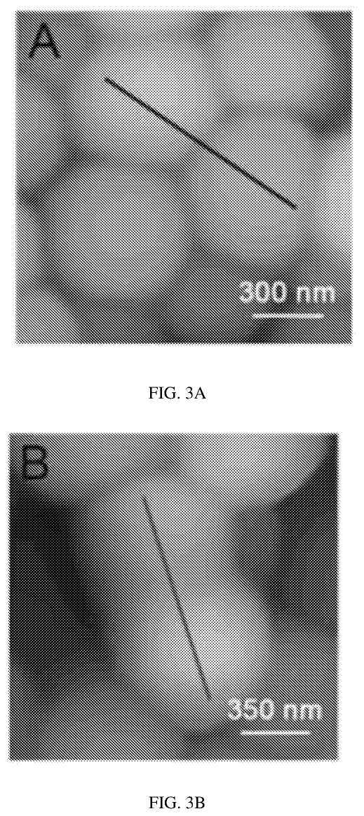

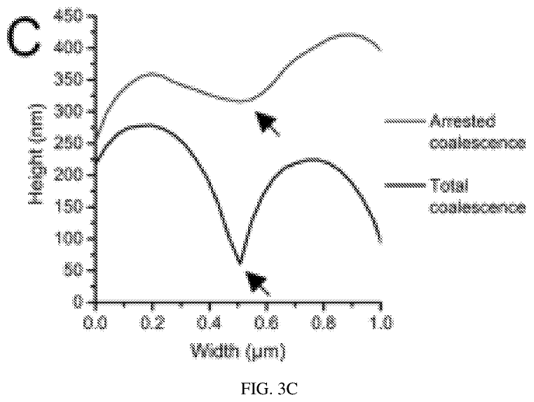

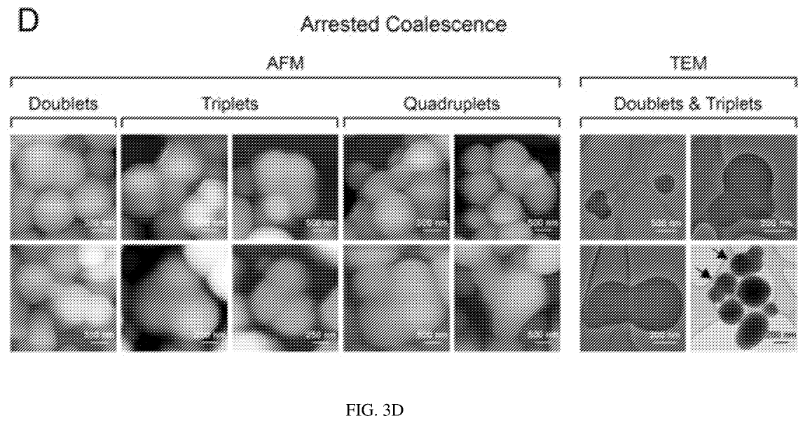

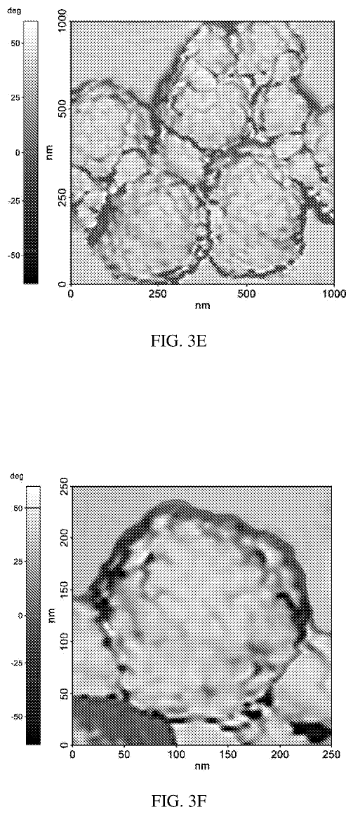

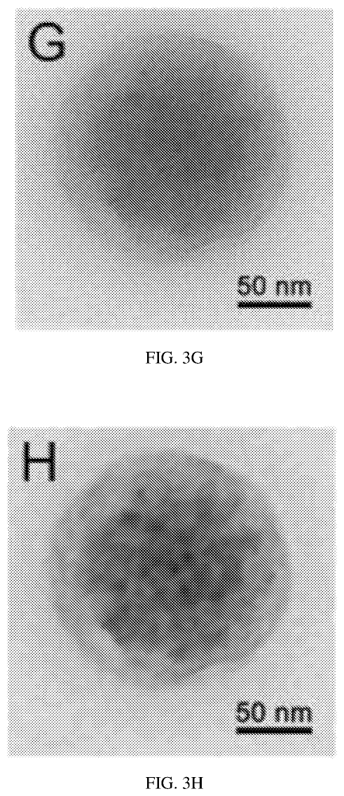

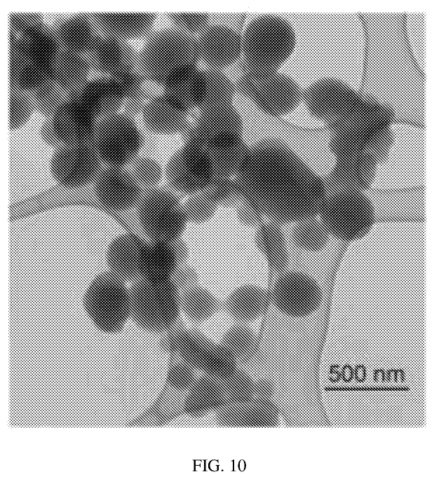

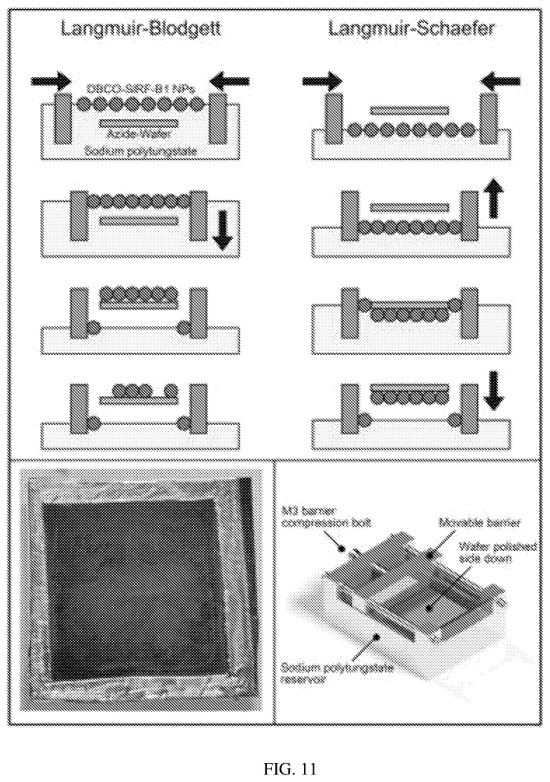

Further evidence of coalescence was observed by the presence of arrested coalescence, causing incomplete merger. Using AFM, different states of arrested coalescence were identified. Coalesced particles had surface profiles showing a sharp boundary between each other (FIG. 3A). In contrast, for near-complete coalescence this sharp boundary was not observed and instead consisted of a smooth transition between neighbouring particles (FIG. 3B). Both profiles are compared in FIG. 3C, with the arrow depicting the boundary between the particles. The complexity of the growth system may result in arrested coalescence of triplets, quadruplets or higher configurations which is seen more frequently during the self-assembly of nanoparticles ≥600 nm. The AFM and TEM images of arrested coalescence are displayed in FIG. 3D. Long chain coalescence necking between multiple particles were also observed by TEM when the coalescence process was halted prematurely within ten minutes by adding 10 mM TRIS buffer pH 7.0 (FIG. 10).

Using AFM and TEM observations, the internal structure of 200 nm nanoparticles were further investigated and it was found that they consisted of smaller globular units of approximately 20 nm as shown in AFM phase images (FIGS. 3E-3F). TEM imaging revealed similar features (FIG. 3G), with post-imaging processing further enhancing the clarity of the structural details (FIG. 3H).

### Example 3: Fabrication of Photonic Structures

Structural colouration having low-angle-dependence is prevalent in nature and is dependent on the dielectric refractive index, colloid diameter, thickness of the structural layers, and lattice distance. The controllable DBCO-SIRF-B1 nanoparticle sizes provided an opportunity to fabricate photonic structures using bottom-up self-assembly techniques such as physical confinement and gravitational sedimentation. Self-assembled photonic structures are either in the form of photonic crystal structures (PCSs) exhibiting long-range order or photonic amorphous structures (PASs) that have only short-range order, resulting in iridescence and structural colouration, respectively.

### Example 4: Langmuir-Schaefer Surface Monolayer Immobilization Method

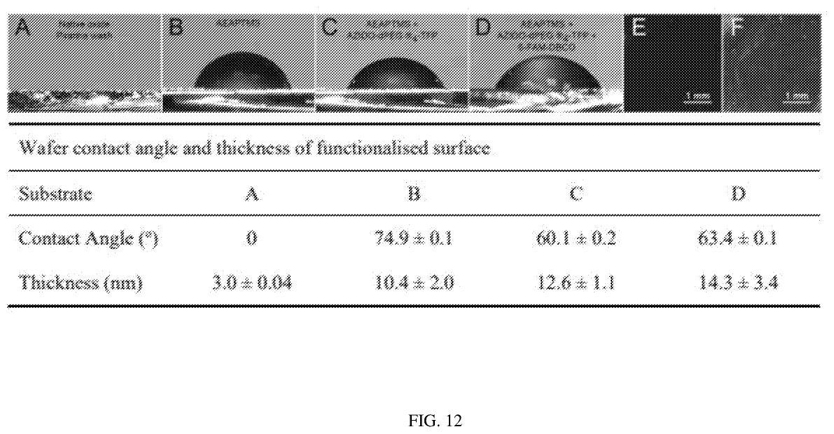

To fabricate either PCSs or PASs using DBCO-SIRF-B1 nanoparticles, the use of Langmuir-Blodgett/Schaefer deposition method was investigated. The concentrated and turbid DBCO-SIRF-B1 nanoparticle suspension was carefully added to the air-water interface required for deposition but sedimented over time their higher mass density. To ensure that the nanoparticles remained at the air-carrier interface, sodium polytungstate, an inert and low toxicity heavy liquid with adjustable solid/water ratio-dependent density (1.0-3.1 g/cm³), was used as the carrier medium instead of water. A customised Langmuir-Blodgett/Schaefer mini device was fabricated using precision CNC machining (FIG. 10), which enabled either the Langmuir-Blodgett or the Langmuir-Schaefer methods to be used for DBCO-SIRF-B1 monolayer deposition. Exploiting the specificity of click chemistry, DBCO-SIRF-B1 nanoparticles was covalently immobilised to silicon wafer surfaces. Aminoalkylsilane [3-(2-Aminoethylamino) propyl] trimethoxysilane (AEAPTMS) was functionalised onto wafers and the free amines were subsequently conjugated with Azido-dPEG®4-TFP ester. Functionalization was confirmed by ellipsometry, contact angle and fluorescent labelling (FIG. 12).

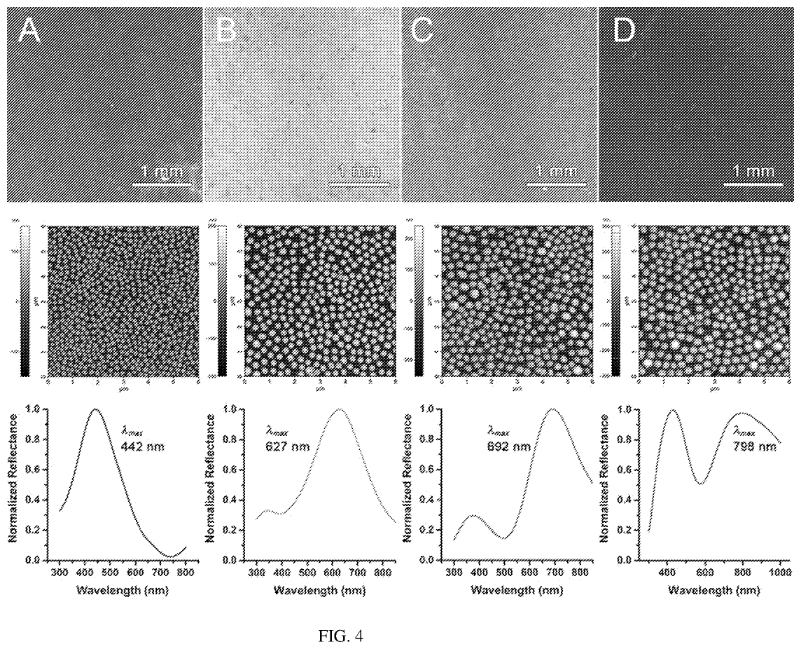

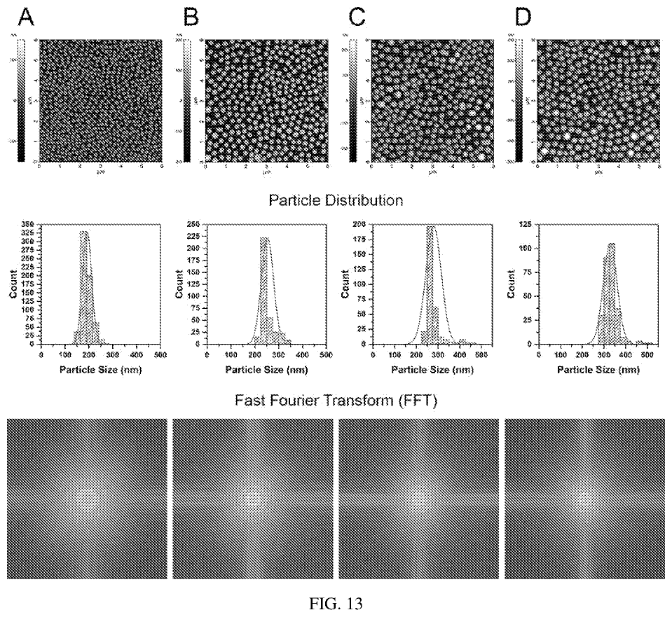

With the Langmuir-Blodgett method, the highly hygroscopic sodium polytungstate formed a very thin layer between the wafer surface and the nanoparticles, resulting in partial monolayer formation. The Langmuir-Schaefer deposition method proved to be superior in fabricating monolayer of DBCO-SIRF-B1 nanoparticles on the wafer surface. Acceptable monolayer Langmuir coatings were also produced from nanoparticle sizes of 170 nm, 240 nm, 270 m, and 310 nm, the theoretical reflectances of blue (λ=442 nm), orange (λ=624 nm), red (λ=702 nm), and near-infrared (2=806 nm) were calculated using the random close-packed volume fraction (f=0.64). All coatings displayed structural colourations as shown in FIGS. 4A-4D (top panel), with the respective AFM images of the coatings, measured reflectance values. The reflectance peak wavelength for amorphous structure can be approximated by the following equation:

$${λ}_{max} = 2 d \sqrt{\frac{2}{3} ( {n}_{eff}^{2} - {sin}^{2} θ )} λ max = 2 dneff \qquad Equation ( 1 )$$

where λₘₐₓ is the maximum reflection wavelength of the particle array, d is the diameter of the DBCO-SIRF-B1 nanoparticles, n_{eff} is the average refractive index of the DBCO-SIRF-B1 nanoparticles, which also accounts for the dielectric-air composite. The average refractive index n_{eff} can be derived using the following equation:

$${n}_{eff} = \sqrt{( {n}_{SlRF - B 1}^{2} \times f ) + ( {n}_{air}^{2} \times ( 1 - f ) )} \qquad Equation ( 2 )$$

where the refractive index of reflectin nSIRF-B1=1.44, nₐᵢᵣ=1, and f is the volume fraction occupied by the nanoparticles. The nanoparticle distribution, volume fraction, and Fast Fourier Transform (FFT) were analyzed using ImageJ (FIG. 13), which confirmed that the particles were quasi-monodisperse and the monolayer assemblies lacked long-range order. Under incident light normal to the surface, structural colouration was observed in all coatings with visualization angles 0 to 35°.

### Example 5: Drop-Cast Immobilisation Method

Temperature is an important factor in thermal-assisted colloidal self-assembly of long-range PCSs with iridescence, as it affects nanoparticles diffusion in the suspension. It was investigated if thermal-assisted colloidal self-assembly (TACSA) could be applied to the DBCO-SIRF-B1 nanoparticles by simply using the drop-cast method to fabricate multi-layered iridescent PCSs at elevated temperatures. Since proteins are susceptible to denaturation at high temperatures, the temperature was limited to a maximum of 60° C. and the effects of TACSA on 215 nm average nanoparticle size were investigated. Samples prepared at 23° C. and 40° C. produced structural colouration, and coating defects were evident at higher temperatures (FIG. 14). No iridescence was observed at any temperature, indicating the absence of long-range PCSs. The investigation was extended to the other different sizes of DBCO-SIRF-B1 nanoparticles derived from FIG. 1D. AFM imaging (FIG. 15) showed that samples prepared ≥40° C. exhibited randomly stacked particles with uncontrolled thicknesses, and with patches of empty spaces 2.5 μm² or larger. Samples prepared at temperature ≤40° C. showed better nanoparticle dispersion across the surface. Larger nanoparticle sizes self-assembled from 30-35% v/v ACN buffer were not significantly affected by temperature.

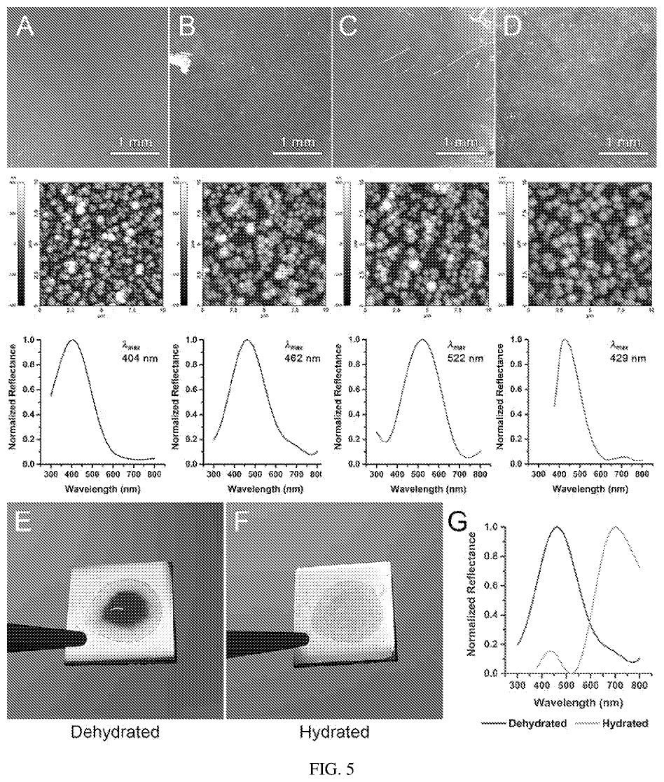

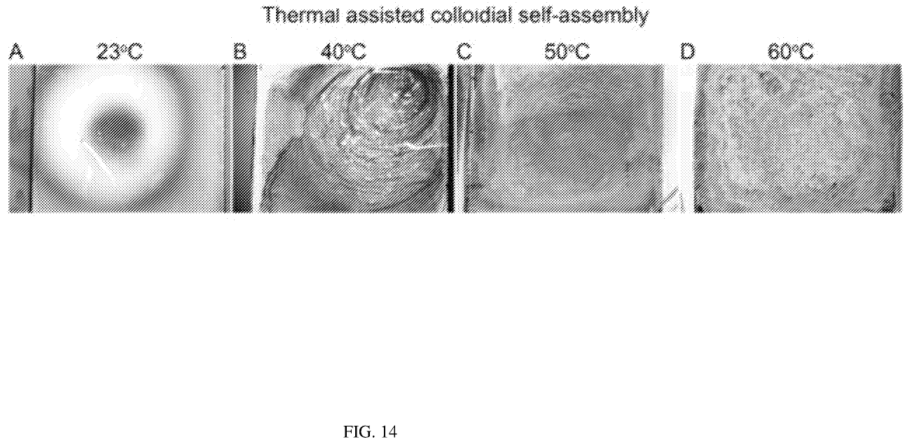

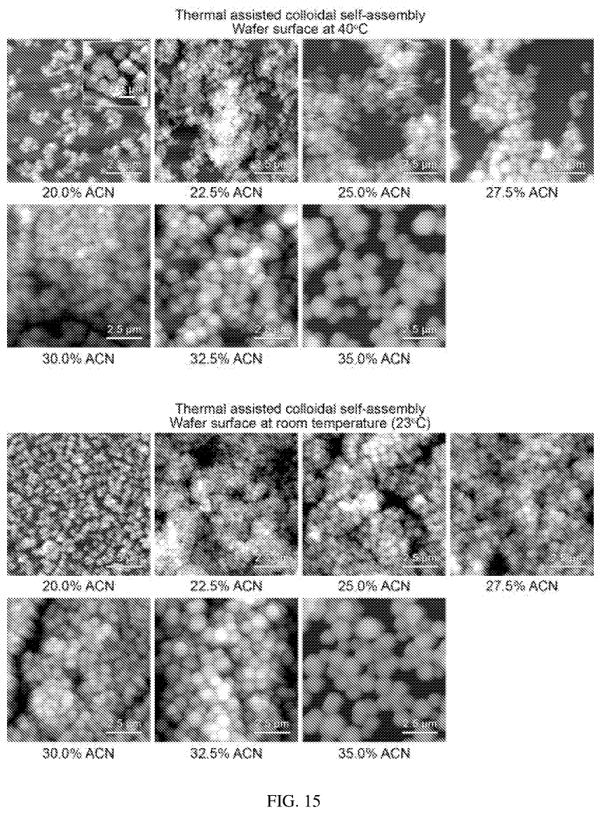

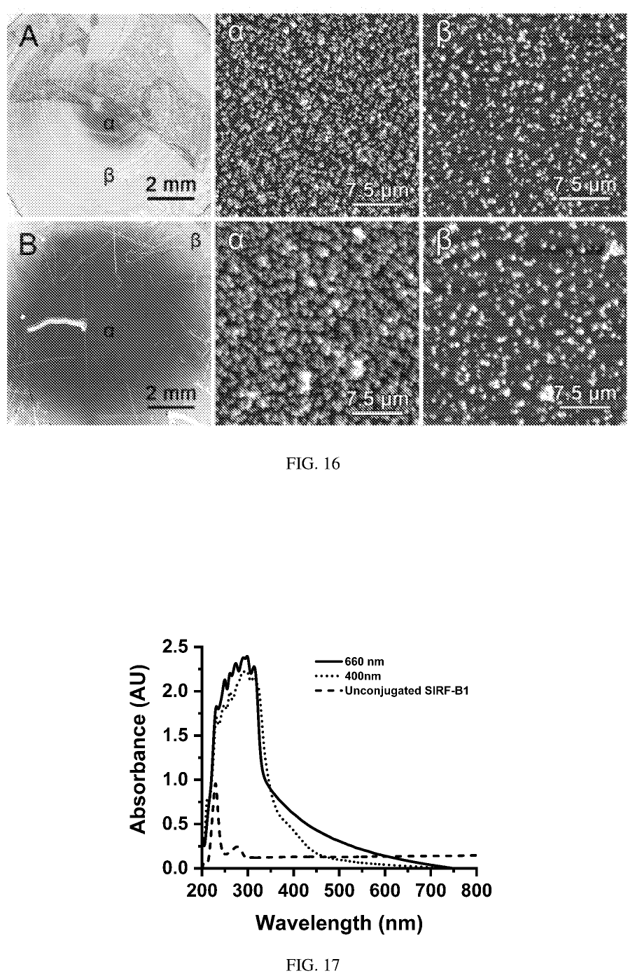

The drop-cast volume was optimised by reducing the volume to 300 μL per 225 mm² of wafer surface area and using a maximum temperature of 35° C. for 400 nm DBCO-SIRF-B1 nanoparticles. Instead of the expected near infrared-red reflectance signature at λ=850 nm based on the Bragg-Snell equation, a violet hue was observed under perpendicular incident light. The experiment was repeated with particle sizes of 400 nm, 460 nm, 520 nm and 660 nm using the optimised conditions, displaying violet, blue, green, and red colouration respectively (FIGS. 5A-5D), except in the periphery of the drop-cast films where no colouration was observed due to wider inter-particle spacing >2.5 μm2 in these regions (FIG. 16). The respective AFM images and measured reflectance values of the coating are shown in FIGS. 5A-5D and AFM Z-heights in FIG. 17. Where structural colouration was observed, AFM imaging showed that the optimised conditions had good nanoparticle surface dispersity, significantly reduced particle stacking (almost monolayer), and an interparticle spacing less than 1 μm. The structural colouration of random close-packing coatings had a stronger reflectance intensity than samples with random loose-packing prepared from drop-cast volumes less than 300 μL.

Finally, there was an interest in triggering a dynamic shift in reflectance, which was hypothesized that it could be achieved by inducing nanoparticle swelling. The blue reflectance film was used (λₘₐₓ=453 nm) and condensed water vapour was applied over the dehydrated monolayer (FIG. 5E). The colour spontaneously red-shifted to 700 nm (FIG. 5F) as monitored with the spectrometer (FIG. 5G). Upon gradual evaporation of the surface moisture, the spectra blue-shifted back to its original spectrum and this process could be repeated multiple times in quick successions without any changes to the sample's surface or spectrum peak. Under incident light normal to the surface, structural colouration was observed in all coatings with visualization angles between 0 to 45°.

The observable structural coloration when nanoparticles have diameters equivalent to visible wavelength is due to the incoherent scattering effect through isotropic photonic pseudoband gaps. Interestingly, this effect is similar to cephalopods' pigment granules found in their chromatophores whose size (ca. 500 nm) is of the same magnitude as visible wavelengths. The absence of wavelength absorption in reflectin nanoparticles coupled with their random close-packed monolayer arrangements results in single particle (resonator) scattering characteristics that are dependent on the individual nanoparticle size, shape, refractive index, and volume fraction of the scatterers.

### Producing Red Coloration Coatings by Absorbance of Secondary Resonant Peak.

Despite the measured wavelength of 700 nm on the hydrated monolayer, there was not any form of red structural coloration observed. Models proposed in the literature explained the challenges to achieve saturated red structural color in amorphous photonic structures, which is attributed to the higher-order resonant mode (secondary resonant peak) shifting into the blue wavelength and intensifying as the particle size increases. Since the blue wavelength has a stronger optical confinement in the resonator, the leaky resonance of findings coincide with the observations indicating that saturated red coloration in the hydrated film was absent (FIG. 5F, FIG. 5G), whereas blue and green colors were not affected since the secondary resonant peaks are in the UV region. Chromatophores in cephalopod skins are able to exhibit deep red color, because they contain ommochrome pigments, including xanthommatin and decarboxylated xanthommatin. Since the light absorbance of xanthommatin pigments peaks at 430 nm, it effectively removes the secondary higher-order resonant peak from the blue spectrum, making the red color dominant. It was targeted to mimic the removal of this secondary resonant blue peak from the 660 nm nanoparticle coating, so that the sample would scatter in the red wavelength (600-700 nm). As shown in FIG. 5D, the secondary resonance peak at 429 nm was present. While hydrogel or metal core-shell coatings have been designed to weaken or eliminate this secondary higher-order resonance peak by matching the refractive index of the nanoparticles' boundaries to that of the surroundings, the goal was to mimic cephalopods' use of absorbing molecules, since protein nanoparticles are soft biomaterials and have limited processing conditions for core-shell fabrication.

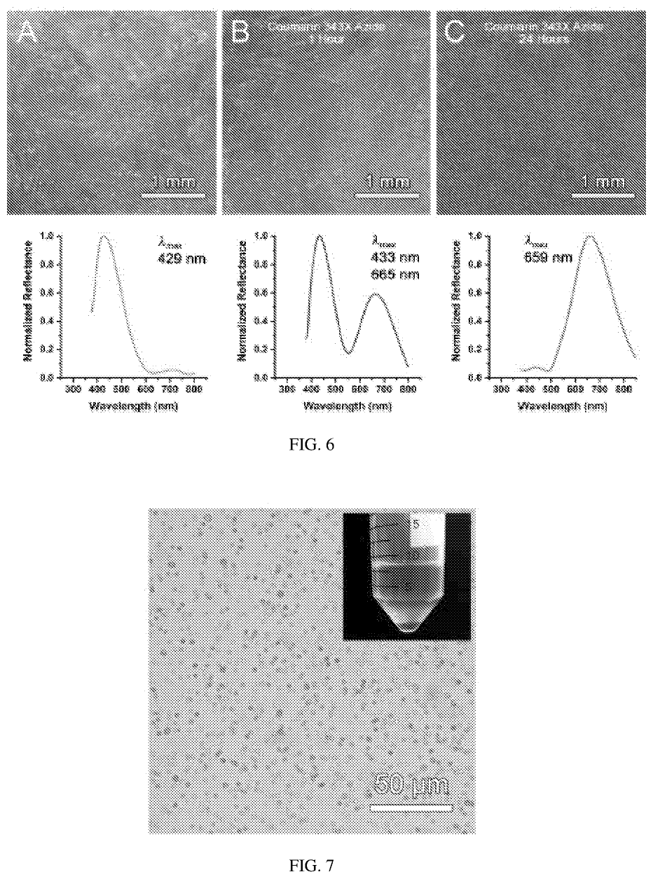

To this end, the fluorescent molecule coumarin 343× azide was explored, which effectively absorbs in the blue wavelength near 430 nm. Using the same click chemistry principle to conjugate the reflectin nanoparticles to the wafer substrate, coumarin 343× azide (1 mM in dimethyl sulfoxide, 50 μL drop-cast) was conjugated to the 660 nm nanoparticle coating (shown in FIG. 4D) for at least 4 h to investigate if red structural coloration could be achieved. Coumarin 343× azide should have a negligible effect toward increasing the particle size, since the nanoparticle surface would only be a molecule thicker. The structural coloration and reflectance measurements before (FIG. 6A) and after conjugation (FIG. 6B and FIG. 6C) confirmed that the 430 nm secondary resonance peak was significantly suppressed after 24 h, resulting in the red color becoming dominant. When the conjugation time was less than an hour, the sample appeared purple, which was due to the mixture of blue and red wavelengths from partial conjugation. This was verified by reflectance measurements (FIG. 6B). Since coumarin 343× azide was used, there is a possibility that the fluorescence emission wavelength may cause unwanted peaks in the reflectance spectra. However, no characteristic emission peak between 477 and 520 nm was detected.

TABLE 3
Summary of Measured Peak Reflectance of Visible Wavelength
Equivalent Reflectin Nanoparticle Size and Its Distribution with
Respective Full Width at half maximum (FWHM) of the Spectrum

| | Targeted | | | | λ | |
|---|---|---|---|---|---|---|
| Targeted | size | ACN | DLS | AFM | Measured | FWHM |
| Color | (nm) | (%) | (nm) | (nm) | (nm) | (nm) |
| Violet | 400 | 23.0 | 408 ± 29 | 398 ± 53 | 404 | 210 |
| Blue | 460 | 24.5 | 467 ± 32 | 465 ± 57 | 462 | 186 |
| Green | 520 | 25.5 | 524 ± 28 | 517 ± 56 | 522 | 185 |
| Red (with | 660 | 28.0 | 671 ± 37 | 667 ± 54 | 659 | 200 |
| Coumarin | | | | | | |
| 343X Azide | | | | | | |
| conjugation) | | | | | | |

The reflectance FWHM values of the 400-660 nm reflectin nanoparticle coatings were relatively consistent at approximately 180-200 nm as summarized in Table 3. Furthermore, they were not dependent on the nanoparticle size unlike the increase in FWHM reflectance values for the coatings made with 170-310 nm nanoparticles. The broadening of FWHM as nanoparticle size increases has been attributed to the leakage of bound photons from the resonator. Since the photons of higher-order resonant modes are bound more tightly to the resonating nanoparticles, and even more so with blue resonance, this results in sharper spectral response (smaller FWHM values). From the data in FIG. 4 and FIG. 5, it can be inferred that the reflectance spectra for the 400-660 nm nanoparticles coatings is secondary resonance, which redshifts from 170 to 310 nm nanoparticles with increasing intensity, explaining the narrower FWHM. This is further supported by the fact that the near-infrared reflectance at λ=1000 nm for the 400 nm nanoparticle size was significantly attenuated and violet at 2=404 nm dominated the spectrum. If the reflectance of the 170-310 nm nanoparticles coatings are assumed are primary resonances, whereas the reflectance of the 400-660 nm nanoparticle coatings are secondary resonances; the resonance peak in FIG. 5D would be tertiary mode, which has a FWHM of 130 nm. Finally, it has also been shown that structural coloration can still be generated from polydisperse nanoparticles at the expense of multiple scattering peaks. The structural coloration would thus be based on the weighted average of the particle size distribution. Considering this, the quasi-monodisperse 400-660 nm nanoparticles coatings having a standard deviation within 15% were able to generate structural coloration with good FWHM despite not having perfectly monodisperse sizes.

Cephalopods' use of naturally occurring xanthommantin occurs through a careful selection of organic molecules that specifically absorb the secondary higher-order resonance mode at ca. 430 nm. Although reflectin nanoparticles were originally identified in iridophores and responsible for iridescence, they have also been recently discovered in chromatophores, together with other structural proteins including S-Crystallin and r-opsin. It has been suggested that the high refractive index and aggregation-inhibiting S-Crystallin protein, with a high affinity to xanthommatin, functions as a light scatterer. The data show that self-assembled reflectin nanoparticles are also capable of functioning as a light scatterer.

### Discussion

It was able for the first time to modulate the growth of reflectin-based nanoparticles with quasi-monodispersity by simply varying the post-purification dialysis conditions. The self-assembly and conjugation method is based on simple colloidal chemistry with a process carried out at room temperature (23° C., 60% RH), pressure and physiological conditions (pH 7.0). Furthermore, these DBCO-SIRF-B1 nanoparticles are click chemistry ready, and the surface-modification strategy can be implemented to alter their properties with a wide array of azide-functionalised molecules. The data demonstrate that SIRF-B1 conjugated with DBCO can be self-assembled into nanoparticles of various sizes with controlled size distribution, a process that does not depend on the presence of the repeated motifs seen in reflectin A1. The formation of large particles also allowed to shed light on the coalescence behaviour of proteins and to unravel the self-assembly mechanism of reflectins. The method was applied to recombinantly-expressed and purified Doryteuthis pealeii Reflectin A1 (GenBank: FJ824804, with 6× His-tag) and nanoparticle self-assembly was achieved, but a complete systematic study was not carried out.

The colloidal self-assembly experimental conditions did not lead to long-range periodic PCSs; instead the quasi-monodispersed nanoparticles self-assembled into photonic amorphous structures. The Langmuir Schaefer is useful for monolayer fabrication of small nanoparticle sizes, whereas the TACSA drop-cast is a simpler method to fabricate either long-range periodic lattices or random closed-packed monolayer for nanoparticles larger than 400 nm. For both methods, the DBCO-SIRF-B1 coatings produced vivid structural colouration on silicon wafer when the nanoparticles were arranged with inter-particle distance of less than 1 μm. Interestingly, the structural colouration observed for particle sizes larger than 400 nm suggests that these nanoparticles parallel the behaviour of pigmented chromatophores, where ca. 500 nm granules function as band-pass filters through light absorption and scattering, an effect enhanced by the presence of high refractive index proteins and xanthommatin. Hence, the DBCO-SIRF-B1 nanoparticles with average size on the same scale as visible wavelength might have partially mimicked the structure and function of a chromatophore granule.

Coating prepared with click chemistry immobilization remained stable for more than one year at room temperature without any special storage. It is appealing that the single molecule ligand DBCO-Sulfo NHS ester was able to initiate controllable nanoparticle growth, allowing to reveal time-resolved self-assembly of reflectin nanoparticles. Overall, this work provides a broader platform for protein-based photonic structures and may be expanded to other fields such as nano-carriers for controlled drug delivery applications.

### Commercial Applications of the Disclosure

Defense—mitigate thermal detection for camouflage

Commercial—Building windows to reflect NIR wavelengths, reducing heat build-up and saving energy cost

Industrial—Paints and coatings

Photonics—Structural colouration of nanoparticle coatings for use in optoelectronic displays Cosmetics-coloured nail polish, skin lotions to reflect NIR (sunblocks), or absorb UV (DBCO ligand)

Medical—Drug encapsulation nano-carriers for drug deliveries, and other bioconjugation of therapeutic molecules

### Experimental Methods

### Example 6: Sample Collection

A live Sepioteuthis lessioniana squid was caught off Keppel Bay, Singapore and sedated for at least 30 minutes in a 20 L bucket filled with seawater supplemented with 0.15 M of magnesium chloride. The body was washed twice with Milli-Q water and dissection was carried out on site immediately. Three skin specimens each measuring 3×3 cm from different parts of the mantle were excised with a sterile scalpel, washed twice with Milli-Q water to remove the excess chromatophores and immediately stored in RNAlater solution. It was later kept in −80° C. freezer.

### Example 7: RNA-Sequencing of Sepioteuthis lessoniana Iridophores

The skin of S. lessoniana 100 mg in wet weight was quickly cut into smaller pieces and transferred to a sterile 2 mL tube. For every 100 mg of skin tissue, 1 mL of Trizol solution was added. The mixture was vortex thoroughly for 5 min and left to incubate at room temperature for another 5 min. Two methods can be employed to shred the skin tissue. A) The tissue was sonicated on ice with 20% power for 3-5 cycles, 1 s pulse at 50% duty cycle. Insoluble material was centrifuged at 15,000 rpm for 10 min. The supernatant was transferred to a new sterile 2 mL tube. B) The incubated skin tissue in Trizol was transferred to a sterile bead-beater tube and filled with 0.5 mm zirconia beads to half the tube volume, or until the solution nearly reaches the brim of the tube. The vial was placed in the beadbeater and operated at maximum speed for 30 s, after which the vial was cooled on ice for 1 min. This step was repeated 2-3 times.

The lysate from either method A or B was transferred to QiaShredder and spun at 15,000 rpm for 2 min and the flowthrough transferred to a new sterile 2 mL tube. Chloroform 200 μL in volume was added to every 1 mL of Trizol and vortexed for 30 s. The mixture was incubated at room temperature for 5 min and centrifuged at 15,000 rpm for 15 min. The top fraction aqueous phase was transferred to a fresh 2 mL and 1 volume of freshly prepared 70% ethanol was added and gently mixed with the pipette.

The solution was transferred to an RNeasy mini kit column and centrifuged at 15,000 rpm for 1 min and the flow-through was discarded. Solution RW1 700 μL was added to the column, centrifuged for 1 min and the flow-through discarded. Solution RPE 500 μL was added to the column, centrifuged for 2 min and the flow-through discarded. This RPE step was repeated once. The column was transferred to a new sterile collection tube and spun dry for another 1 min. Water which are DEPC treated or RNase-free 40 μL in volume was carefully added to the column, incubated for 1 min and centrifuged at 15,000 rpm. The extracted RNA was stored in −80° C.

The Poly A selection of mRNA was done by DynaBeads Oligo dT and subsequently sequenced using Illumina compatible NEXTflex™ Rapid Directional RNA-Seq kit according to manufacturer's protocol.

### Transcriptome Assembly

The pooled libraries were sequence on a HiSeq 2000 with 2×151 read length. The raw fastq reads were checked with FastQC (http://www.bioinformatics.babraham.ac.uk/projects/fastqc/) and quality trimmed with Trimmomatic. The paired end reads were then pooled and a denovo transcript assembly was performed with Trinity. To estimate the expression level of each transcript, each library was then analysed individually against this reference using RSEM.

### Protein Expression

The plasmid encoding the gene of Sepioteuthis lessoniana reflectin B1 (SIRF-B1) was purchased from Genscript (New Jersey, U.S.A). The pET-28a (+) plasmid was conferred with kanamycin resistance but not encoded with 6× His-tag. Restriction site was selected to be NcoI and XhoI. The plasmid was transformed into BL21 (DE3) E. coli and protein expression was induced by the T7 promoter (Isopropyl β-D-1-thiogalactopyranoside, IPTG). Tartoff Hobbs Terrific broth was used for the sustained growth of E. coli with slight modification where glycerol was increased to 10 mL per litre and potassium phosphate buffer was adjusted to pH 7.4. A 25 mL preculture was supplemented with 50 μg/mL of kanamycin and grown overnight at 37° C. for 16 h.

The 25 mL preculture was centrifuged at 5000×g for 5 min and the supernatant was discarded. The bacteria pellet was resuspended in 10 mL of fresh Terrific Broth and added to 1 L of Terrific Broth culture supplemented with 50 μg/mL of kanamycin and 100 μL of Antifoam 204. The bacteria was cultured for an additional 6 h at 37° C. Recombinant protein expression was induced with 1 mM IPTG for the next 16 h. The 1 L bacteria culture was harvested by centrifuging at 20,700×g for 5 min at 4° C. (Hitachi Koki Himac CR22N, Tokyo, Japan). The cell pellet was resuspended in 50 mL of ice cold 50 mM HEPES pH 7.4 lysis buffer and supplemented with 1 mM PMSF and 10 mM DTT just prior to cell lysis. Cell lysis was carried out using a micro-fluidiser (M110P, Microfluidics International Corporation, Massachusetts, U.S.A) at 20,000 psi for three passes.

The lysed cells were centrifuged at 20,700×g for 5 min at 4° C. and the inclusion bodies were resuspended in 20 mL of ice-cold lysis buffer with addition of 1 mM DTT. This washing step was repeated three times, followed by once with 1% w/v CHAPS zwitterionic surfactant and 10 mM DTT. The suspension was vortexed briefly and left to incubate for 10 min on ice. Three more washing step with only the lysis buffer was repeated to remove the excess surfactant and the inclusion bodies were solubilised in 15 mL of 6M N,N′-Dimethylurea only at 50° C. for 1 h in an ultrasonic bath with occasional brief vortex. The solution was centrifuged at 20,700×g for 30 min at 4° C. and the clarified supernatant was transferred into a 10 kDa regenerated cellulose dialysis membrane. Excess length of the dialysis membrane is required for at least twice the volume increase. The supernatant was dialysed against only Milli-Q water for 24 h with water changes every 3 h. The soluble SIRF-B1 protein was lyophilised for at least 48 h, purged with argon gas and stored at −20° C.

### Optical Microscopy

Light microscopy was performed on the Zeiss A1 upright microscope. Coacervates were imaged in light transmission mode. Coacervates suspension was pipetted onto a clean glass slide with a piece of coverslip, and magnification was set to 50× using the EC “Epiplan-Neofluar” 50×/0.8 HD objective lens. Structural colouration of DBCO-SIRF-B1 nanoparticles immobilised onto silicon wafers were imaged in reflected light mode with Epi Brightfield, and magnification was set to 2.5× using the EC “Epiplan-Neofluar” 2.5×/0.06 HD objective lens. All images were captured at full resolution (2584×1936 pixels) with the Zeiss AxioCam MRc5 5MP Colour Microscope Camera attached to a 60N-C ⅔″ 0.63× C-Mount camera adapter.

### Dynamic Light Scattering (DLS)

### Size

Nanoparticle sizes were determined by Malvern Panalytical Zetasizer Nano ZS and data was analysed using Zetasizer software v8.01. Acquisition settings was set to backscatter angle of 173°, 3 measurement repeats with each measurement having 10 acquisitions lasting 5 s at 25° C. The acquired data for each set is repeated three times from each batch and then from three different dialysis batch. This was to ensure batch to batch reproducibility and stability of the nanoparticles in the buffer. The samples were pipetted into disposable UV micro cuvettes (Cat #759200).

Size Growth of SIRF-B1 with Addition of DBCO

The growth of SIRF-B1 nanoparticle with the addition of DBCO-Sulfo-NHS ester was monitored for a total of 800 s. A total of 80 measurements were recorded, each acquisition lasting 2 s with a delay of 5 s between each measurement. The particle size at time t=0 s was measured with SIRF-B1 nanoparticles after centrifugation and 500 μL was pipetted into the cuvette. Minimal volume of dialysis buffer was used to dissolve 5 mM (1.33 mg) DBCO-Sulfo-NHS ester and subsequently added to the cuvette. Homogenous mixing was done by rapidly pipetting the solution twice followed by analysis. Particle Number Mean was used for data plotting and three additional data point at 1800 s (30 min), 3600 s (1 h) and 7200 s (2 h) were included to extrapolate the data. Curve fitting was performed on the scattered plot in OriginPro 2016.

### Zeta-Potential

The zeta-potential measurement of the nanoparticles is an important and measureable indicator of the colloid stability in dispersions. A high positive or negative (≥+30 mV) zeta-potential confers stability to the particles due to charge repulsion which resist aggregation. The samples used the DTS1070 cuvette where its electrophoretic mobilities were measured and converted to zeta-potential using the Smoluchowski's formula. The number of runs were fixed at 50 repeats with no delay between three set of measurements.

### Strong Cationic Exchange HPLC (SCX-HPLC)

Strong cation exchange buffer is listed in Table 4. Ultrapure urea is dissolved in Milli-Q water and equilibrated to room temperature. Deionisation of the 8M urea solution was carried out for at least 1 h in accordance with manufacturer's protocol using either AG 501-X8 or Bio-Rex MSZ 501 (D) mixed bed resin (Bio-Rad Laboratories, California, U.S.A.) contained in a nylon tea bag. The resin bag was removed and buffered with MES to pH 6.0 with piperidine. The solutions in Table 4 were vacuum degassed through a Corning® 0.22 μm PES bottle top vacuum filter (Ø 45 mm neck, Part #431118) into a vacuum safe amber glass bottle. This solution is stable at room temperature for 1 week. Lyophilised SIRF-B1 protein was weighed and dissolved in SCX buffer A to make a stock concentration of 30 mg/ml supplemented with 10 μL of β-mercaptoethanol (1% v/v) and heated at 85° C. until the protein fully solubilised. The solution was centrifuged at 21,500×g for 10 min at room temperature (Hitachi Koki Himac CT15E, Tokyo, Japan) and syringe filtered through a 4 mm 0.45 μm regenerated cellulose membrane. Purification was carried out on a semi-preparative 5 μm, 1000 Å Polysulfoethyl ATM column (PolyLC Inc., Maryland, U.S.A.). Flow rate was set to 2 mL/min and UV detector was set to acquire chromatogram at 254 nm and 280 nm wavelength. Buffer gradient was set from 0% A to 20% B in 30 min. Maximum sample injection was 15 mg per 500 μL. Collected fractions were pooled together and kept at 4° C.

TABLE 4
SIRF-B1 strong cation exchange parameters

| | MES | | | | Time | Gradient |
|---|---|---|---|---|---|---|
| | free acid | Urea* | NaCl | pH** | (min) | (%) |
| Buffer A | 20 mM | 8M | — | 5.9 | 0 | 0 |
| Buffer B | 20 mM | 8M | 2M | 5.9 | 30 | 20 |

*Deionised urea
**pH adjusted with piperidine

### Sodium Dodecyl Sulfate—Polyacrylamide Gel Electrophoresis and Matrix-Assisted Laser Desorption/Ionization Time-of-Flight

Electrophoresis was carried out using the neutral PAGE system according to manufacturer's protocol. MALDI-ToF was carried out using the sandwich method with sinapic acid as previously described.

Self-Assembly of Sepioteuthis lessoniana Reflectin B1 Protein

Buffers containing 10 mM MOPS at pH 7.0 and varying amount of acetonitrile (HPLC grade) were prepared accordingly based on the desired nanoparticle size. The 10 mM MOPS free acid powder was first added to Milli-Q water, followed by the addition of the required acetonitrile volume. The buffer is homogeneously mixed on a magnetic stirring plate at 200 rpm for at least 1 h for equilibration to room temperature. Sodium hydroxide was added to the buffer until pH 7.0 and finally topped up to a final volume of 1800 mL with Milli-Q water. Three millilitres of HPLC purified SIRF-B1 was pipette into a 3.5 kDa regenerated cellulose dialysis membrane and dialysed against either of the seven buffers for 16 h at room temperature with continuous stirring at 200 rpm. The dialysed protein was transferred to sterile microcentrifuge tubes and centrifuged at 2,500×g for 5 min at room temperature. The clarified supernatant was carefully transferred to new sterile microcentrifuge tubes. All buffer volumes were measured with a measuring cylinder.

Conjugation of Self-Assembled SIRF-B1 with Click-Chemistry Ligand Dibenzocyclooctyne-Sulfo-NHS Ester

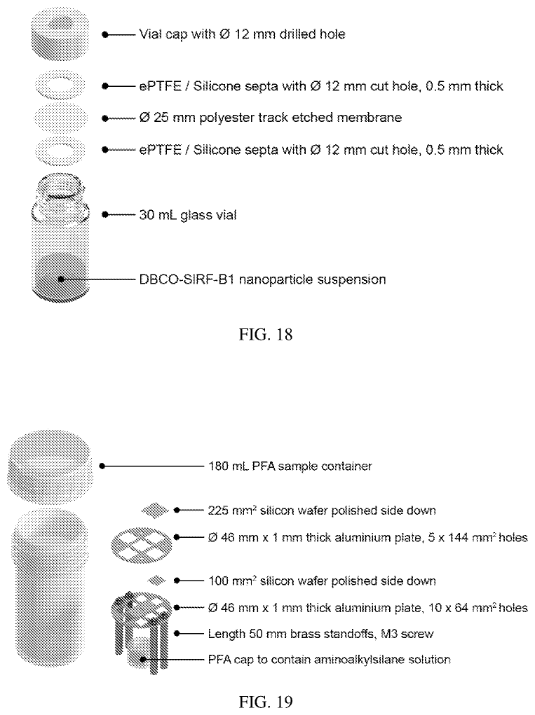

Dibenzocyclooctyne-Sulfo-NHS ester (DBCO-Sulfo-NHS ester) was used as the ligating molecule for the self-assembly of SIRF-B1 at a concentration of 5 mM, lower concentration did not stabilise the particles (they aggregated after a few hours), whereas excess ligand (10-20 mM) did not further affect the size and stability of the nanoparticles. The reaction was left to react for 2 h, forming a turbid yet opalescent yellow-orange solution indicating the presence of nanoparticles. Excess and unreacted DBCO-Sulfo-NHS ester was desalted by dialysing against the same dialysate buffer used to self-assemble the particular SIRF-B1 size. For sample volume of 5 mL to 30 mL, a self-made setup was fabricated (FIG. 18) to utilise 25 mm diameter, GVS or Oxyphen (Unique-Mem/Rotrack) track-etched polyester membranes with pore size ranging from 200-1000 nm. Dialysis and protein concentration were achieved by gravity filtration by inverting the vial, with completion time within 1 h. (Table 5). The use of large pore size membranes allows rapid dialysis and simultaneously filter small sized nanoparticles which improved the overall monodispersity.

For desalting of small sample volumes between 1-3 mL, Merck Millipore Ultrafree®-MC Durapore® PVDF centrifugal filter may be used instead of dialysis. Desalting was carried out three times by adding same volume of fresh buffer filtered through the Whatman Anotop 0.02 μm syringe filter (same dialysate buffer used to self-assemble the desired SIRF-B1 size). Alternatively, the sample may be desalted using a 10 kDa regenerated cellulose dialysis bag for 16 h. The 5.0 μm filter was used for sample cleanup (centrifuged at 10×g for 30 s; resuspend and repeat) if there are dust particulates or minor aggregates.

TABLE 5
Track-etched filter membrane selection for particle size and desalting time

| | | Volume 3-30 mL (Vial) | | Volume 1-3 mL | |
|---|---|---|---|---|---|
| | | Track Etched | Desalting/ | Centrifugal Filter | Sample |
| Acetonitrile | Particle | Membrane | concentration | Durapore ® | clean up |
| (%) | size (nm) | pore (μm) | time (Minutes) | PVDF (μm) | (optional) |
| 10.0 | 173.2 ± 24.3 | 0.1 | 60 | 0.1 | Durapore ® |
| 12.5 | 184.4 ± 27.2 | 0.1 | 60 | 0.1 | 5 μm |
| 15.0 | 197.6 ± 28.5 | 0.1 | 60 | 0.1 | |
| 17.5 | 215.7 ± 23.6 | 0.1 | 60 | 0.1 | |
| 20.0 | 258.4 ± 21.1 | 0.2 | 40 | 0.22 | |
| 22.5 | 369.4 ± 27.6 | 0.2 | 40 | 0.22 | |
| 25.0 | 505.3 ± 25.3 | 0.4 | 40 | 0.45 | |
| 27.5 | 635.9 ± 21.6 | 0.6 | 20 | 0.45 | |
| 30.0 | 777.5 ± 24.9 | 0.6 | 20 | 0.65 | |
| 32.5 | 892.5 ± 67.1 | 0.8 | 20 | 0.65 | |
| 35.0 | 946.3 ± 90.3 | 0.8 | 20 | 0.65 | |

### SURFACE Functionalisation of SiO₂ Substrate with [3-(2-Aminoethylamino) propyl|trimethoxysilane and Azido-dPEG®4-TFP ester

Silicon P-type wafer with intrinsic native oxide of approximately 3 nm were cleaved to 100 mm² or 225 mm² and first cleaned in 5% v/v Decon90 detergent for 5 min with sonication, followed by anhydrous toluene, 2-propanol and Milli-Q water twice each. The cleaned wafers were then immersed in 40 mL of piranha solution (sulfuric acid-30% hydrogen peroxide ratio 3:1) for 1 h contained in a PFA container. The wafers were rinsed thoroughly with Milli-Q water and dried on a hotplate at 80° C. for 1 min. Wafers are freshly prepared for each experiment and used immediately.

Cleaned silicon wafers were placed facing down onto a 5 cm tall self-made platform with 8 mm or 12 mm square holes supporting the wafers (FIG. 19). A 20 mm diameter PFA screw cap containing 500 μL of [3-(2-Aminoethylamino) propyl] trimethoxysilane (AEAPTMS) was placed at the bottom of a 180 mL PFA container. The hand tightened container was placed in a preheated oven at 130° C. for 16 h. The AEAPTMS coated substrates were sonicated in anhydrous toluene, 2-propanol and Milli-Q water twice each. For immediate use, the wafer was dried on an 80° C. hotplate for 1 min.

Wafers functionalised with AEAPTMS was reacted with 10 mM Azido-dPEG®4-TFP ester in the incubation buffer consisting of 10 mM HEPES and 150 mM NaCl at pH 8.0 with DMSO to buffer ratio of 9:1. It was left to react for at least 4 h at room temperature in a sealed PFA container. The substrate was sonicated with anhydrous DMSO and Milli-Q water twice each for 5 min and dried on an 80° C. hotplate for 1 min and used immediately.

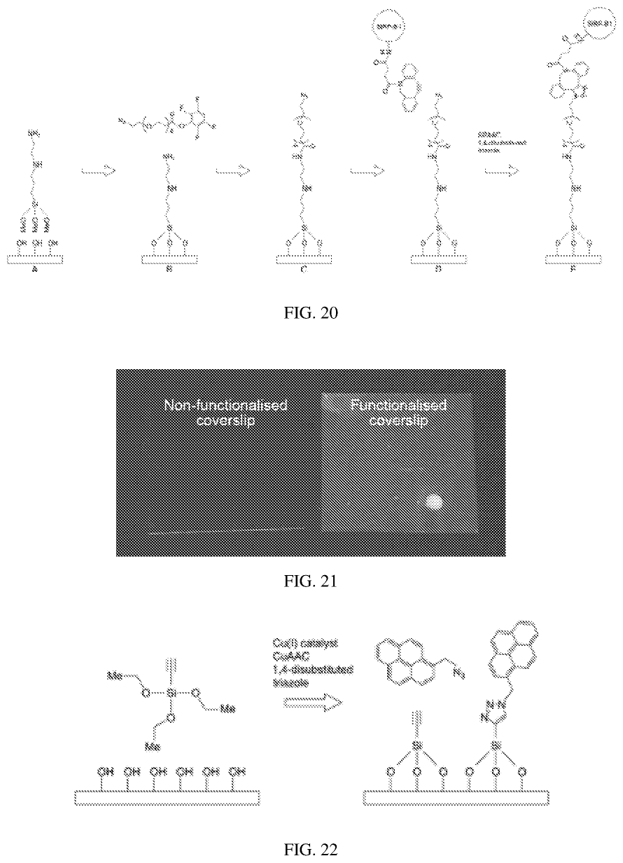

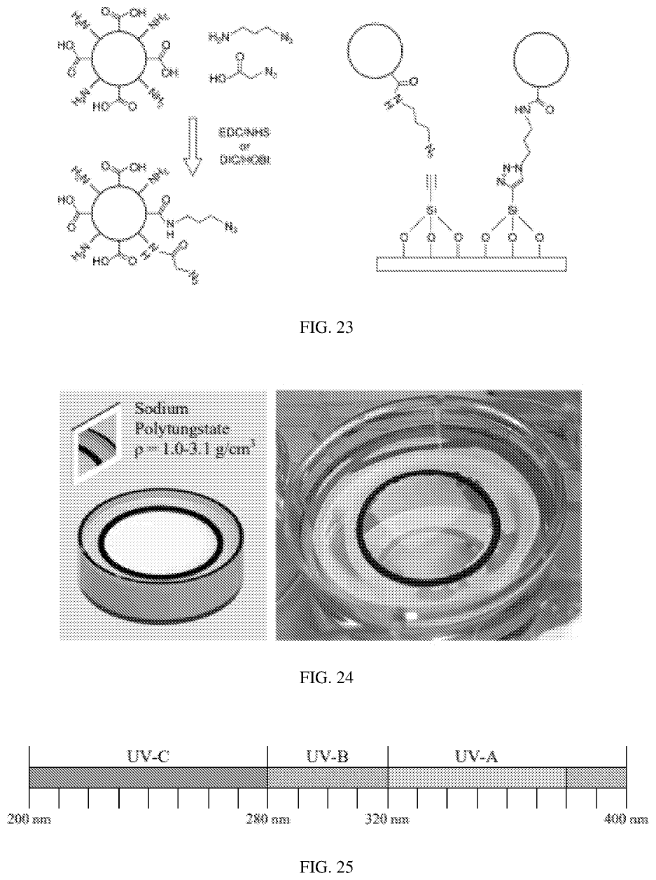

Azide-functionalised wafers were confirmed with 6-FAM-DBCO as the labeling fluorescent probe. The fluorescent probe 500 μM in 10 mL was first dissolved in 1 mL of anhydrous DMSO and subsequently added to 9 mL of 10 mM HEPES, 150 mM NaCl pH 8.0 buffer. The substrate was incubated in the solution at room temperature for 16 h overnight in the dark. The substrate was sonicated in 50% DMSO-water three times for 1 min each and air-dried. A schematic reaction scheme is depicted in FIG. 20.

Fluorescence microscopy was carried out with a Nikon microscope and imaged with a Nikon DS-Ri2 CMOS camera through a Plan Fluor 4×/0.13 WD 16.5 objective lens. The imaging software used was NIS Elements D v4.5 (Build1117). Laser wavelength for FITC 488 nm was selected for the excitation of 6-FAM-DBCO fluorescent probe. Exposure was set to 5 s with an analogue gain of 64×.

### Transmission Electron Microscopy

Nanoparticle dispersed solution 4 μL in volume was pipetted onto a TEM copper grid with Ultrathin C Film on Lacey Carbon support film (Ted Pella Product number 01824) and left to sit for 5 min before excess sample solution was removed by absorption with a piece of filter paper. Imaging was carried out using JEOL 2010 TEM with an Ultra High Resolution (UHR) pole piece, equipped with a Gatan 794 MSC CCD and operated at an acceleration voltage of 200 kV.

### Langmuir-Schaefer Deposition Method for DBCO-SIRF-B1 Nanoparticles

A custom computer numerical control (CNC) machined Langmuir-Schaefer deposition trough was fabricated using aluminum grade AA 5083 for small wafers up to 225 mm². The internal edges of the trough reservoir were lined with PTFE tape. Sodium polytungstate was used as the carrier medium, and the density of the solution was fixed at 2.8 g cm 3. Sodium polytungstate weight of 22.97 g was added to 5.04 g of water (total volume 10 mL) and homogeneously mixed with a magnetic stirrer. The pH was then adjusted with 6 M NaOH until pH 7.0. For each experiment, 1.2 mL of sodium polytungstate was added into the trough reservoir, followed by the addition of 60 μL nanoparticle suspension (10× concentration). The ACN was allowed to evaporate for at least 30 min before the trough is compressed. The Kibron Force Sensor KBN320 (Kibron Inc., Helsinki, Finland) was used to monitor the interfacial tension (surface pressure) of the Langmuir film using a DyneProbe (perimeter 1.59 mm). The compression area isotherm was plotted using the values. An azide-functionalized wafer was masked with a polyester sticker or aluminum tape along the edge with a square cutout. The vacuum supported wafer (Pisco Ø 8 mm vacuum pad (VPB8PFS-4B) connected to a 12 V KnF micro gas diaphragm pump (NMP830KPDC-B-HP)) was lowered using an Edmund Optics XYZ manual stage (Stock #36-034) until it touched the nanoparticle surface. The click reaction was carried out for 16 h, after which it was submerged into Milli-Q water for at least 1 h, rinsed with Milli-Q water, and air-dried.

### Drop-Cast Method for DBCO-SIRF-B1 Nanoparticles

The as-prepared DBCO-SIRF-B1 nanoparticles were drop-cast onto 225 mm² azide-functionalised wafer with a volume of 300 μL. The wafer was warmed to 35° C. and the sample solution was homogenously mixed by pipetting very gently every 1 h. This step was performed when the settled nanoparticle forms a cross shape on the wafer surface which prevented proper monolayer self-assembly. The entire process takes about 6 h and mixing should stop when the cross shape is no longer observable. This is also the time when the sample meniscus angle is almost zero. The sample solution was allowed to dry out for 24 h and after which it is submerged into Milli-Q water for at least 1 h, rinsed with Milli-Q water and air-dried.

### Atomic Force Microscopy

The nanoparticle coatings were imaged using Parks NX10 AFM (Parks System, Suwon, Republic of Korea) equipped with a NanoWorld Pointprobe NCSTR probe. Imaging was carried out in noncontact mode with a desired scan area of 1-30 μm2 and an image size of 512×512 pixels with a scan rate of 0.25 Hz under ambient conditions. Image analysis and processing was done in XEI 4.3.4.Build22. The tip deconvolution estimation was performed in the software for accurate measurements on the X and Y axes. In brief, an AFM calibration standard with highly defined pitch of 300 nm was used. Based on the manufacturer's recommendation, Z-height would not be accurate, as the probe tip may not reach the base of the calibration standard. The calibration image was processed with the AFM software, and tip estimation was performed using the data. This data is stored and applied to images scanned with the same probe.

### Ellipsometry

Molecule layer thickness that are deposited consecutively onto the wafer were measured using J. A. Woollam VASE® ellipsometer controlled by WVASE32 v3.77 software. The refractive index of the material was taken from published literatures or approximated using known properties of the molecules. The refractive index of AEAPTMS, Azido-dPEG®4-TFP and 6-FAM-DBCO were approximated to be n=1.444, 1.454 and 1.816 respectively. The unknown layer thickness of the nanoparticle coating on the substrate was loaded with the Cauchy.mat layer. The thickness and refractive index of the Cauchy layer was fitted and lowest Mean Squared Error (MSE) values between 1-20 were used.

### Reflectance Measurements

Reflectance data were measured using Avantes Avaspec ULS2048 spectrometer with grating from 200 nm to 1100 nm attached to the Zeiss A1 upright microscope. The fibre optic end is attached to a 60N-C ⅔″ 0.63× C-Mount camera adapter. Zeiss HAL 100 microscope lamp was used as the light source. The infrared-red filter on the lamp housing was removed for measurements at near infrared-red wavelength up to 900 nm. Spectrometer software version used is Avasoft© 8.12.0.0. Calibration was performed using the calibration tile WS-2.

### Method Variations

The following summary describes the other changes in experimental parameters which led to nanoparticle formation. The changes in these parameters had been carried out with the click chemistry ligand Dibenzocyclooctyne-sulfo-NHS-ester during experimental method screening, unless otherwise stated explicitly. Other method parameters on surface functionalization and Langmuir-Schaefer set up is included here. The parameters described herein are not optimised, and there are no complete systematic studies to contribute the data as reliable and usable. The parameters described here offer alternatives which may be probable for future uses after optimisations.

### Nanoparticle Formation

### 1. Variation of Organic Solvent

The organic solvent can be replaced with alcohols (HPLC grade), with ethanol and 2-propanol tested with 5-15% concentration in 5-20 mM (normally 10 mM) MOPS, MES or HEPES buffer at pH 7.0 to pH 7.4 buffers, and may be extended to 15-50% organic solvent concentration. Formation of nanoparticles were induced, but investigation into the effects of varying alcohol concentration was not carried out completely as seen with acetonitrile.

2. Variation of Buffer pH with Varying Organic Solvent Concentration

The SIRF-B1 self-assembly buffer was varied between pH 4.0 to pH 10.0 using 10 mM Good's buffering agent, with varying acetonitrile concentration between 5-50%. Nanoparticle size could be varied, but size control is unpredictable and nanoparticle stability is not guaranteed.

### 3. Full Aqueous Buffers

The self-assembly of SIRF-B1 can be accomplished in pure water, or in aqueous buffers varied between pH 8.0 to pH 10.0 using 10 mM of any Good's buffering agent, or 5-50 mM sodium borate (normally 10 mM) or imidazole (50-200 mM), with the addition of sodium chloride (0-150 mM) for ionic charge screening, and additionally with the use of either one of the 2 types surfactants listed in section: 3.1 Buffers with surfactants.

3.1 Buffers with Surfactants

The self-assembly of SIRF-B1 had been carried out with 2 types of surfactants, either with zwitterionic surfactant 3-[(3-Cholamidopropyl) dimethylammonio]-1-propanesulfonate (CHAPS), or neutral surfactant n-Octyl-β-D-glucopyranoside in 0.5-10 mM concentration. Surfactants may not work well with buffers containing organic solvents.

3.2 Buffer with Anti-Oxidant

Aqueous buffer was added with 1-10 mM ascorbic acid as an antioxidant and buffering agent at pH 6.0, titrated with sodium hydroxide (as sodium ascorbate) or piperidine to pH 6.0, and additionally with the use of either one of the 2 types surfactants listed in section: 3.1 Buffers with surfactants. This method was without the use of Dibenzocyclooctyne-sulfo-NHS-ester, but can encompass the use of it as pH 6.0 is the lower limit of NHS ester reaction.

3.3 Buffer with Other Organic and Inorganic Acid

Aqueous buffer with 1 mM acetic acid or 0.61 mM phosphoric acid at pH 4.0 had been tested, and additionally with the use of either one of the 2 types surfactants listed in section: 3.1 Buffers with surfactants. This method was without the use of Dibenzocyclooctyne-sulfo-NHS-ester, and may not work well with NHS ester reaction.

### 4. Nanoparticle Formation in Organic Solvent

SIRF-B1 nanoparticle formation below the size of 200 nm were synthesized using Methyl-BCyclodextrin (MBCD), without the use of Dibenzocyclooctyne-sulfo-NHS-ester. Purified SIRF-B1 protein with a concentration of 1 mg mL 1 was pipette into a 3.5 kDa regenerated cellulose dialysis membrane, and dialyzed against water only. Protein to MBCD ratio of 1:1, 1:2, 1:4 (w/w) were investigated. No nanoparticles were detected at ratio of 1:6, 1:8 and 1:10. The samples were inverted a few times until the MBCD were dissolved and then lyophilized for 24 h.

Anhydrous ethyl acetate 1 mL in volume was added to the lyophilized sample and sonicated for 1 min to resuspend the protein nanoparticles. Aggregates were centrifuged at 5000×g for 20 min at 4° C. The supernatant was carefully transferred to a new Eppendorf tube and centrifugation was repeated again once with the same parameters.

Ratio of SIRF-B1:MβCD in Ethyl
Acetate in relation to particle size

| | Particle | Polydispersity |
|---|---|---|
| Ratio | Size (nm) | index (PDI) |
| 1:1 | 659 ± 24.1 | 0.544 |
| 1:2 | 85.3 ± 6.08 | 0.183 |
| 1:4 | 104.6 ± 11.4 | 0.262 |

5. Conjugation of SIRF-B1 Nanoparticle with Other Click Chemistry Ligand

Nanoparticle self-assembly had been tested with the following click chemistry ligand listed in the following Table 6 which was taken from Table 2 other than Dibenzocycloocytne-sulfo-NHS ester. The ligands had been tested on all the experimental condition described Section 1, 2 and 3 in place of Dibenzocycloocytne-sulfo-NHS. The term “dPEG” in Azido-dPEG4-TFP (product number 10567) is Quanta BioDesign's acronym for “discrete polyethylene glycol” or “discrete PEG”, indicating single molecular weight PEG technology.

TABLE 6
Ligands screening with SlRF-B1 nanoparticles

| Ligand | M.W. | Solubility |
|---|---|---|
| Azidoacetic NHS Ester | 198.1 | Water, DMSO |
| Azidoacetic NHS Ester | 198.1 | Water, DMSO |
| Azidobutyric NHS Ester | 226.2 | Water, DMSO |
| Azido-PEG₃-NHS Ester | 344.3 | Water |
| Azido-PEG₄-NHS Ester | 388.4 | Water |
| Azido-dPEG ®₄-TFP | 439.4 | DMSO, DMF |

### Surface Functionalization

Surface functionalization had been successfully accomplished using Azido-dPEG4-TFP on the solid substrate and Dibenzocycloocytne-sulfo-NHS ester on the nanoparticle, and covalent immobilisation between them occurs via copper-free strain promoted Strain-promoted azide-alkyne cycloaddition. Before this, copper assisted click chemistry (CuAAC) had been initially investigated, as well as other functionalization schemes detailed below. The following functionalization methods can be applied to any material surface that can be hydroxylated. Surface functionalization with amines using APTES is described here, and the same procedures may be applied to functionalised carboxylic surface. Selection of the carboxylated or aminated ligands and its derivatives would simply be in reverse.

### Stabilising Reflectin B1 Nanoparticles

Purified reflectin B1 (SIRF-B1) self-assembles in acetonitrile buffers at pH 7.0, and subsequently self-assembles into controlled and quasi-monodisperse nanoparticles with the addition of the click chemistry ligand DBCO-Sulfo-NHS ester.

The nanoparticles undergo sedimentation after a period of time, where nanoparticles larger than 500 nm settle within a day, and nanoparticles smaller than 500 nm settle within 2 to 5 days. Settled and compacted reflectin nanoparticles are unable to resuspend homogenously in the same buffer even after sonication. This is partly caused by hydrophobic aggregation of the DBCO ligand on the nanoparticles despite having a zeta-potential of −38 mV (borderline stable).

The nanoparticles also aggregate in solution after dialysis against full aqueous buffers, such as DMEM buffers used in keratinocytes cell uptake studies where acetonitrile must be removed. To mitigate aggregation, the DBCO attached to reflectin B1 can be end capped with an azide hydrophilic ligand.

Three ligands were selected for conjugation to the DBCO-SIRF-B1 nanoparticle listed below:
Azido-PEG₃-OH
Azido-PEG₃-phosphonic acid
Azido-PEG₂-sulfonic acid

It should be noted that end capping the DBCO on the SIRF-B1 nanoparticles with the above-mentioned ligands effectively inhibits further conjugation or modifications to the surface chemistry.

Two nanoparticle sizes of 360 and 500 nm were tested, where each of the ligand (1 μL or 1 mg) was added to 500 μL of the nanoparticle suspension. The click chemistry reaction was allowed to proceed at 40° C. for 4 hours. Once the nanoparticle suspension has cooled, it was transferred to a 3.5 kDa regenerated cellulose membrane and dialysed against DMEM buffer pH 7.4 for 16-24 hours.

Nanoparticle suspension dialysed against DMEM without the conjugation (1) of any of the above-mentioned ligands precipitated. Majority of the settled nanoparticles could not be resuspended by gentle agitation or sonication.

Nanoparticle with the ligands (2) shows insignificant aggregation. Majority of the settled nanoparticles could be resuspended by gentle agitation.

(1) The aggregates were centrifuged at 2,500×g for 10 mins and the supernatant was dialysed against water for 16-24 hours and lyophilised. The non-aggregated protein was only 30% or less.

(2) The aggregates were centrifuged at 2,500×g for 10 mins and the supernatant was dialysed against water for 16-24 hours and lyophilised. The non-aggregated protein was 90% or more (minimal loss).

The screening results below in Table 7 are preliminary and should only be taken as a reference. Results can even fluctuate beyond the reading in the table above due to the presence of residual aggregates which still requires optimisation for removal. The negatively charged phosphonic acid and sulfonic acid induced charge repulsion and imparted hydrophilicity from its PEG arm, mitigating aggregation in full aqueous buffers and it was analysed that the overall nanoparticle size decreased. After dialysis and the removal of acetonitrile, the nanoparticles have increased water uptake causing it to swell (acetonitrile dehydrates the nanoparticles).

The ligands are not limited to the above-mentioned molecules. Longer PEG arm linker from PEG₄ to PEG₃₆-OH can be further tested. Although phosphonic and sulfonic do not have longer PEG arms, testing with carboxylic end groups with varying PEG arm length also can be screened.

TABLE 7
The nanoparticle size and zeta potential were analysed by
DLS before and after DMEM dialysis,
with and without PEG ligands.

| | Before dialysis | After dialysis | Zeta-potential |
|---|---|---|---|
| 360 nm | 366 ± 26 nm | 460 ± 37 nm | −38 mV |
| DBCO-SlRF-B1 + | | | |
| Azido-PEG₃-OH | | | |
| 500 nm | 507 ± 32 nm | 600 ± 51 nm | −38 mV |
| DBCO-SlRF-B1 + | | | |
| Azido-PEG₃-OH | | | |
| 360 nm | 344 ± 38 nm | 380 ± 43 nm | −45 to −60 mV |
| DBCO-SlRF-B1 + | | | |
| Azido-PEG₃- | | | |
| phosphonic acid | | | |
| 500 nm | 473 ± 47 nm | 523 ± 64 nm | −45 to −60 mV |
| DBCO-SlRF-B1 + | | | |
| Azido-PEG₃- | | | |
| phosphonic acid | | | |
| 360 nm | 339 ± 41 nm | 386 ± 43 nm | −45 to −60 mV |
| DBCO-SlRF-B1 + | | | |
| Azido-PEG₂- | | | |
| sulfonic acid | | | |
| 500 nm | 467 ± 55 nm | 537 ± 57 nm | −45 to −60 mV |
| DBCO-SlRF-B1 + | | | |
| Azido-PEG₃- | | | |
| sulfonic acid | | | |

1. Surface Functionalization with (3-Aminopropyl) Triethoxysilane (APTES)

Transparent glass slide and coverslip were first treated with piranha solution (sulfuric acid-30% hydrogen peroxide ratio 3:1) and functionalised with aminoalkylsilane (3-Aminopropyl) triethoxysilane (APTES).

### 1.1 APTES Functionalization Via Solution Method

Concentration of APTES in anhydrous acetone was 3 mM and a clean cover slip was immersed in the solution for 3 hours. The coverslip was washed in acetone and methanol twice each and annealed in the oven at 150° C. for 16 hours. Concentration of APTES in anhydrous ethanol was 10% v/v and a clean cover slip was immersed in the solution for 15 minutes. The coverslip was washed in ethanol five times and annealed in the oven at 150° C. for 16 hours. Concentration of APTES in anhydrous toluene was 2% v/v and a clean cover slip was immersed in the solution for 3 hours. The coverslip was washed in toluene and methanol twice each and annealed in the oven at 150° C. for 16 hours.

### 1.2 APTES Functionalization Via Vapour Deposition Method

### 1.2.1. Dilute Vapour Deposition

Concentration of APTES in anhydrous toluene was 1% v/v and a clean cover slip was suspended in a 180 mL PFA bottle using Kapton tape. The dilute APTES solution was added to a small metal cap and place into the PFA bottle. The bottle was purged with argon gas and placed in the oven at 150° C. for 16 hours.

### 1.2.2. Concentrated Vapour Deposition

A clean cover slip was suspended in a 180 mL PFA bottle using Kapton tape. APTES 50 μL in volume was added to a small metal cap and place into the PFA bottle. The bottle was purged with argon gas and placed in the oven at 150° C. for 16 hours.

It was observed that vapour deposited APTES coated glass slides have a contact angle of approximately 80-90°, whereas solution based APTES coating varies between 40-60°. As amine itself is hydrophilic, the lower contact angle suggests that more amine are populated on the surface. Although most literature had consistent results with vapour deposition, the high contact angle could mean that the APTES molecule is oriented sideways, exposing more of the alkyl chain, or that the amine is buried below the surface as it has a high propensity to bind itself to the hydrophilic silanol group.

### 2. Amide Formation Between Aminoalkylsilane and Ethynyl or Propargyl Functional Group Via Carboxylic-Amine Coupling

Ethynyl or propargyl functional group bearing carboxylic group can be coupled to amine using standard DIC/HOBt or EDC/NHS chemistries. Below are two molecules which had been tested: 4-ethynylbenzoic acid (hydrophobic molecule); Propiolic acid (hydrophilic molecule) 4-ethynylbenzoic acid 2 mM in concentration was dissolved in 10 mL of anhydrous N, N-dimethylformamide, followed by the addition of 2.5 mM N,N′-Diisopropylcarbodiimide (DIC) and 3 mM Hydroxybenzotriazole (HOBt). The reaction was carried out under argon for 30 minutes. The APTES coated glass was immersed in the solution and left to react at room temperature under argon for at least 6 hours.

Propiolic acid 20 mM in concentration was dissolved in 1 mL of 5 mM MES, 0.5 M NaCl, pH 6.0 buffer, followed by the addition of 30 mM 1-Ethyl-3-(3-dimethylaminopropyl) carbodiimide (EDC) and 40 mM N-Hydroxysuccinimide (NHS). The reaction was carried out under argon for 15 minutes. The coupling solution is added to 29 mL of 5 mM HEPES buffer, 0.15 M NaCl pH 7.2 with an immersed APTES coated glass and left to react at room temperature under argon for at least 6 hours.

### 3. Copper (I)-Catalyzed Alkyne-Azide Cycloaddition of Ethynyl or Propargyl to Azide Functional Group

1-Azidomethylpyrene is used here in the methods as an example, and can be replaced with any azide conjugated samples or molecules. The cycloaddition utilised the copper ligand trishydroxypropyltriazolylmethylamine (THPTA) as the catalyst. Biological samples containing carboxylic acid and/or amines may be conjugated with azidopropylamine (for carboxylic acid) or azidoacetic acid (for amines), or its equivalent through the use of EDC/NHS (aqueous) or DIC/HOBt (non-aqueous) chemistries. This method however must be optimised to prevent activated carboxylic acid on one nanoparticle from reacting with amine on another nanoparticle. This can cause aggregation.

1-Azidomethylpyrene at 0.4 mM concentration for 10 mL was first dissolved in 1 mL of 2 parts anhydrous N,N-dimethylformamide and 1 part Milli-Q water. Anhydrous copper sulfate 0.5 mM, copper ligand tris-hydroxypropyltriazolylmethylamine (THPTA) 0.5 mM and sodium ascorbate 5 mM was weighed and added to the PFA container. Nine millilitres of 2 parts anhydrous N,N-dimethylformamide and 1 part Milli-Q water was added to the container and thoroughly mixed to dissolve the solids by repeated pipetting. An ethynyl or propargyl functionalised APTES glass was immersed into the solution followed by the addition of the 1-Azidomethylpyrene solution. The container was purged with argon and capped tight. The reaction was carried out at room temperature for at least 2 hours with stirring at 200 rpm. Anhydrous N,N-dimethylformamide may be substituted with anhydrous DMSO.

3. Simplification of Coupling with Triethoxy (Ethynyl) Silane

The aminoalkylsilane APTES may be replaced with triethoxy (ethynyl) silane, skipping the method outlined using 4-ethynylbenzoic acid and propiolic acid as the intermediate molecule. Reaction scheme is depicted below using 1-Azidomethylpyrene as an example. Functionalisation of triethoxy (ethynyl) silane to hydroxylated surface is similar to that of APTES in solution and vapour deposition method.

### Monolayer Coating of SIRF-B1 Nanoparticles on Functionalised Wafer

Another simple method of monolayer coating using only minimal material was earlier conceived with idea and reference from the following publication: Ryan van Dommelen, Paola Fanzio, Luigi Sasso, Surface self-assembly of colloidal crystals for micro- and nano-patterning, Advances in Colloid and Interface Science, Volume 251, 2018, Pages 97-114, https://doi.org/10.1016/j.cis.2017.10.007. The density of the sodium polytungstate solution could be adjusted between ρ=1.0-3.1 g/cm³. To enable Langmuir-Blodgett method, the density of the substrate needs to be higher than the density of the prepared sodium polytungstate solution. For Langmuir-Schaefer, the density of the substrate needs to be lower than the density of the prepared sodium polytungstate solution. This can be applied to any substrate. The density of any proteins, independent of its molecular weights had previously been determined to be between 1.22-1.43 g/cm³, thus the density of sodium polytungstate solution needs to be at least 1.5 g/cm³. An FKM O-ring (1.85 g/cm³) or generic nitrile O-ring (1.00 g/cm³) was floated on the sodium polytungstate solution. FKM O-ring is preferred as the nanoparticle solution contains acetonitrile. The nanoparticle solution was slowly added into the O-ring and contained within it. Acetonitrile was allowed to evaporate for at least 30 min. The amount of nanoparticle to be added needs to be experimentally determined using this method. An azide functionalised wafer was masked with a polyester sticker frame along the edge and very gently placed within the O-ring. The masking restricts movement of the nanoparticles during the click chemistry reaction from vibrations, and confines the packed particles within the substrate area. The click reaction was carried out for 24 to 72 h, after which it was submerged into Milli-Q water for at least 1 h, removed and air-dried.

### Toxicity Effects of Reflectin Nanoparticles in Keratinocytes and its UV Absorbing Properties

Introduction Ultraviolet radiation (UV) is abundant in natural sunlight comprising of UV-A, -B and -C (FIG. 25). UV-A and -B is able to bypass the atmosphere and accelerate skin aging as the former is able to penetrate deeply into the dermis layer, whereas the latter causes sun burn. Although UV-C is mostly blocked by the ozone atmosphere, its germicidal properties used in sterilisation lamp poses potential health risks. These UV wavelength causes eye injury (e.g. irritation and inflammation of cornea) and skin injury (e.g. erythema). Chronic exposure to UV radiation can also accelerate the skin aging process and increase the risk of skin cancer.

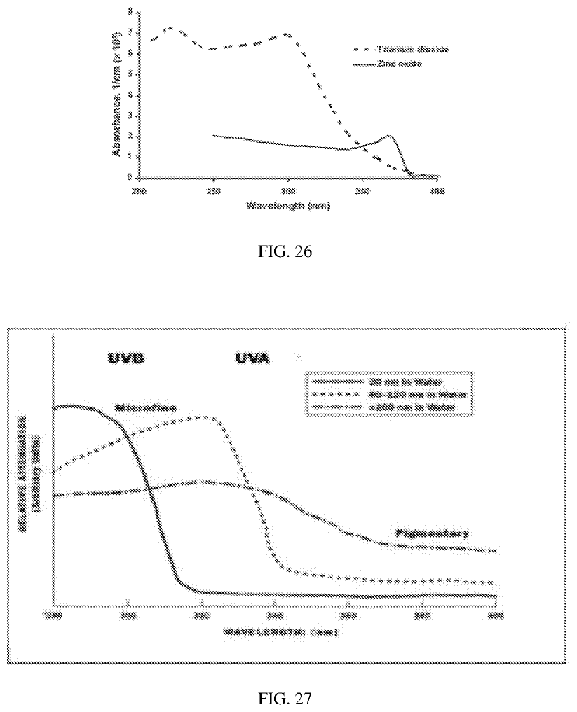

To mitigate the harmful effects of UV from natural and artificial sources, personal care products such as sunscreens with a high SPF number is effective in the absorption of UV radiation (FIG. 26). The active compounds of these sunscreen are either nanosized zinc oxide, titanium dioxide, or a combination of both. However, in the past decade, the extensive use of these metal oxide nanoparticles were found to have some toxicity even when applied externally on the epidermis, although it has been rebutted that the compounds are safe to use at concentrations found in the commercial products, generally between 1-3%. The uptake of these metal oxide nanoparticles in keratinocyte cells are suggested to occur when the healthy stratum corneum are compromised. Nonetheless, the production of these nanoparticles in factories inadvertently exposes workers to nanoparticle powders, which is highly damaging to the lungs, bioaccumulation in target organs and prolonged circulation in vivo. Although there has been conflicting research on the toxicity of zinc oxide and titanium dioxide when used in sunscreens, it is still worthwhile to identify a direct replacement to these compounds, which will possibly help to further reduce the exposure of fine powders during production, source for a renewable and sustainable UV absorbing material, and reduce the dependence on titanium metal.

Commercial formulation of zinc oxide and titanium oxide have nanoparticle sizes between 0.1-10 μm, particularly less than 100 nm as smaller sizes absorb the UV wavelength with increased efficiency (FIG. 27).

This report currently tests only the toxicity effect of DBCO-SIRF-B1 on keratinocyte in vitro using ZnO as the control. ZnO nanopowders are commercially available with 130 nm or less, and this preliminary study is thus limited to a narrow range of ZnO nanoparticle size.

In the following section, the UV absorbance and DLS was carried out with TiO₂ nanoparticle controls. Moreover, the toxicity study of DBCO-SIRF-B1 in keratinocyte cells was compared with ZnO control.

### Results

The size of the TiO₂ nanoparticle controls was first analysed using dynamic light scattering (DLS) to determine the hydrodynamic radius. Titanium dioxide nanoparticles (10 μL) was extracted from the commercial sunscreen (Bioré, Japan, SPF 50+) and resuspended in 1 mL of Milli-Q water. The results are shown in Table 8.

TABLE 8
Nanoparticle size of the controls and commercial
sunscreen was analysed using DLS

| | TiO₂ nanoparticle controls | | | Biore |
|---|---|---|---|---|
| | 21 nm | 200 nm | 300 nm | Sunscreen |
| DLS (Z-Avg) | 109 ± 15 nm | 183 ± 4 nm | 337 ± 6 nm | 152 ± 13 nm |

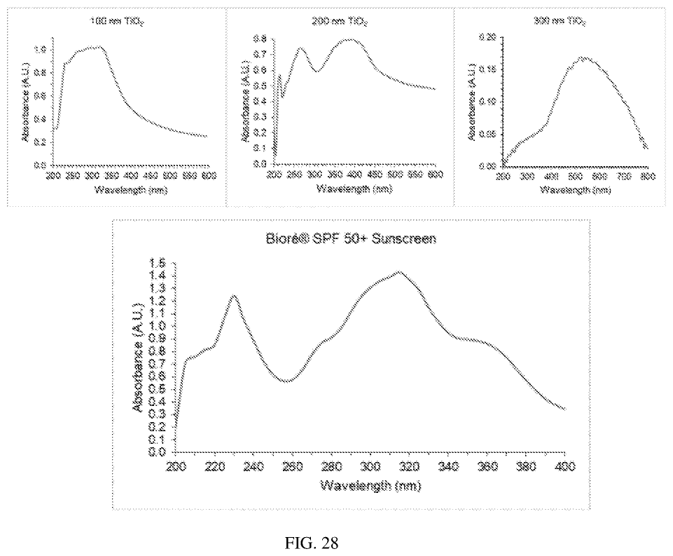

The UV absorbance of the TiO₂ controls and sunscreen was subsequently analysed using UV-Vis spectrometer (Nanodrop2000c) as shown in FIG. 28.

It was observed that as the nanoparticle size increases, the absorbance redshifts into the visible wavelengths. For TiO₂ nanoparticle size above 200 nm, it no longer has any UV absorbing properties, thus for effective UV absorption, the nanoparticle size needs to be at least 200 nm and smaller. It was also noted with nanoparticle increasing size that the absorbance intensity decreases (due to decreased surface area) when compared across with the same concentration.

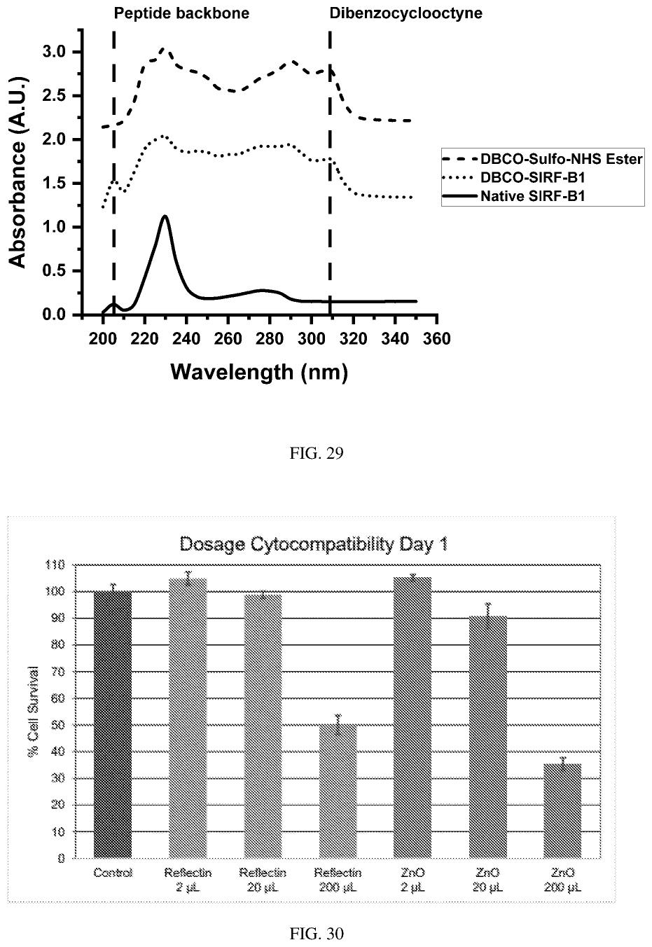

Reflectin nanoparticles conjugated with dibenzocyclooctyne (DBCO-SIRF-B1) has been previously reported to form quasi-monodisperse nanoparticles with controllable size between 170-1000 nm. 400 nm DBCO-SIRF-B1 was synthesized, and its UV absorbance was recorded (FIG. 29). The spectra show similar UV absorbance profile to 100-200 nm TiO₂.

A comparison between unconjugated reflectin nanoparticle DBCO-SIRF-B1 with large DBCO-SIRF-B1 nanoparticle size of 400 and 660 nm is shown in FIG. 17. The UV absorbance is significant in the UV-B and UV-C region.

### Results for Toxicity Effect in Keratinocytes

In the following preliminary study, only zinc oxide was used as the control in comparison with 400 nm DBCO-SIRF-B1. Cell survival % was tabulated and plotted in FIG. 30 after a 1-day cytocompatibility test. 400 nm DBCO-SIRF-B1 shows better tolerance by keratinocytes as compared to ZnO nanoparticle (250-350 nm hydrodynamic radius) of the same concentration. To allow for the cells to properly uptake the nanoparticles, DMEM solution was used without serum. Consequently, the experiment could not progress beyond 2 days due to cell death.

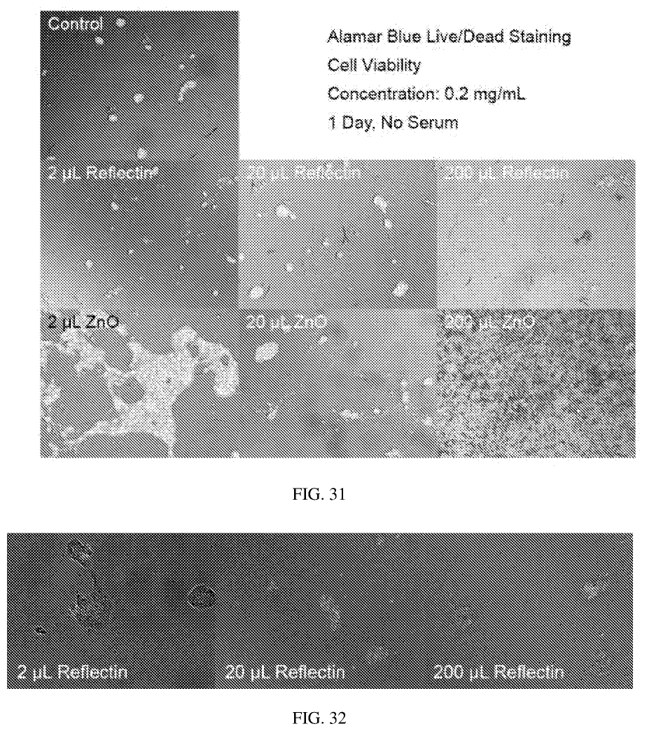

The live/dead alamar blue and 5-FAM azide staining was performed on the cells and imaged as presented in FIGS. 31 and 32.

### CONCLUSION

DBCO conjugated reflectin nanoparticles show promising results which may be further investigated for its use as a replacement for TiO₂ and ZnO nanoparticle formulation in cosmetic and personal care products. First, DBCO-SIRF-B1 offers similar UV absorbance profiles compared to a combination of TiO₂ and ZnO. Second, 400 nm DBCO-SIRF-B1 nanoparticles shows better tolerance and lowered cytotoxicity in keratinocytes compared to 250 nm ZnO.

The current data suggests that the UV absorbance properties of DBCO-SIRF-B1 comes from DBCO itself, and it is independent of the reflectin nanoparticle size. Therefore, more studies will be carried out to determine the toxicity effect of reflectin nanoparticle in keratinocyte cells based on size and concentration of DBCO-SIRF-B1 as compared to the different TiO₂ controls.

### Materials and Methods

### Reflectin Nanoparticles

Zinc oxide nanoparticles with a hydrodynamic radius of 250 nm at a concentration of 0.2 μg mL⁻¹ was used as the control. Reflectin nanoparticle was synthesized with 17.5% v/v acetonitrile (285±18 nm) and dialyzed against aqueous DMEM solution (385±26 nm). Final concentration ca. 0.2 μg mL⁻¹.

### UV Absorbance of Titanium Dioxide Nanoparticles

Ultraviolet absorbance of titanium dioxide controls was analysed with Nanodrop (Nanodrop2000c) between wavelength 200 to 800 nm. Titanium dioxide nanoparticle sizes tested were 21 nm (anantase, Sigma Aldrich), 200 nm (rutile, Nanografi) and 490 nm (rutile, Nanografi). The powders 1 mg in weight were weighed into glass vial and resuspended in 1 mL of Milli-Q water in a sonicator bath for 10 mins.

### UV Absorbance of DBCO-SIRF-B1 Nanoparticles

Ultraviolet absorbance of 400 and 660 nm DBCO-SIRF-B1 nanoparticles (0.3 mg mL⁻¹) were analysed with Nanodrop (Nanodrop2000c) between wavelength 200 to 800 nm. The 400 nm nanoparticle suspension was diluted by 250× with water for improved UV absorbance profile (signal desaturation).

### Seeding Keratinocytes

Keratinocytes (HaCaT passage 20) were seeded with 10% FBS DMEM media on 48-well plate to a cell density of 75000 cells cm 2. The cells were incubated overnight for 16 h at 37° C. with 5% CO2. The seed media was aspirated and washed with serum free media (DMEM). After washing and removing DMEM, controls and the nanoparticle solution was added. Zinc oxide nanoparticles was weighed and freshly prepared using DI water, sterilized with UV for 10 min, diluted to desired concentration, sonicated for 10 mins, exchanged to DMEM buffer and sonicated again.
Reflectin in DMEM: 2, 20, 200 μg mL⁻¹
ZnO in DMEM: 2, 20, 200 μg mL⁻¹

### Control DMEM Solution

Acetonitrile 17.5% v/v was used for this experiment, and it is toxic to cells at this concentration. 3 mL of 17.5% ACN, 82.5% H₂O, 10 mM MOPS at pH 7.0 was dialysed against 500 mL of DMEM solution for 24 h with a 10 kDa regenerated cellulose membrane at 200 rpm. The final acetonitrile concentration in the DMEM solution after dialysis would be 0.35 μL mL⁻¹.

Sample assignment to well plate negative and positive controls, together with reflectin and zinc oxide nanoparticles were assigned to a 48 well plate shown in Table 9.

TABLE 9
Assigned sample in the well plate. Three different
concentrations with triplicates.

| | 1 | 2 | 3 | 4 | 5 |
|---|---|---|---|---|---|
| A | Blank | 0 μg mL⁻¹ | 2 μg mL⁻¹ | 20 μg mL⁻¹ | 200 μg mL⁻¹ |
| | Media | Solution control | Reflectin | Reflectin | Reflectin |
| | no cells | | | | |
| B | Blank | 0 μg mL⁻¹ | 2 μg mL⁻¹ | 20 μg mL⁻¹ | 200 μg mL⁻¹ |
| | Media | Solution control | Reflectin | Reflectin | Reflectin |
| | no cells | | | | |
| C | Blank | 0 μg mL⁻¹ | 2 μg mL⁻¹ | 20 μg mL⁻¹ | 200 μg mL⁻¹ |
| | Media | Solution control | Reflectin | Reflectin | Reflectin |
| | no cells | | | | |
| D | | | 2 μg mL⁻¹ | 20 μg mL⁻¹ | 200 μg mL⁻¹ |
| | | | ZnO | ZnO | ZnO |
| E | | | 2 μg mL⁻¹ | 20 μg mL⁻¹ | 200 μg mL⁻¹ |
| | | | ZnO | ZnO | ZnO |
| F | | | 2 μg mL⁻¹ | 20 μg mL⁻¹ | 200 μg mL⁻¹ |
| | | | ZnO | ZnO | ZnO |

### Legend

A1-C1, blank media
A2-C2, cells with control DMEM solution
A3-C3, 2 μg mL⁻¹ reflectin
A4-C4, 20 μg mL⁻¹ reflectin
A5-C5, 200 μg mL⁻¹ reflectin
D3-F3, 2 μg mL⁻¹ ZnO
D4-F4, 20 μg mL⁻¹ ZnO
D5-F5, 200 μg mL⁻¹ ZnO

The control solution and nanoparticles in DMEM were incubated with the cells for 24 h at 37° C. with 5% CO2. The cells were imaged using brightfield microscopy at 4× magnification. The nanoparticle solution was removed and 200 μL of 1× AlamarBlue in DMEM was added to check for cell viability according to manufacturer's protocol (Thermofisher product DAL1025). This was incubated for 1 h and 100 μL of assay solution was analysed with the plate reader with excitation 560 nm and emission 590 nm. The mean results were calculated by:

Mean {(X ug/mL value−blank no cell value)/[mean (0 μg/mL value-blank no cell value)]}*100%.

The assay solution was discarded, and the nanoparticle solution was added to the cell and incubated for 24 h. After which, the nanoparticle solution was removed and 1 well was stained using 200 μL of live dead, while another well was stained for DBCO-SIRF-B1 nanoparticles using 5-FAM azide fluorescent probe. Live dead staining done by manufacturer protocol (https://ibidi.com/img/cms/support/AN/AN33_Live_Dead_staining_with_FDA_and_PI.pdf) Hoescht done in tandem with live dead, by manufacturer's protocol (https://www.thermofisher.com/sg/en/home/references/protocols/cell-and-tissueanalysis/protocols/hoechst-33342-imaging-protocol.html)

### Cell Fixation and Fluorescein Staining

The cells were fixed with 4% paraformaldehyde for 1 h and washed thrice with PBS buffer. 0.1% Triton X-100 was added for cell permeabilization and washed thrice with PBS buffer. 5-fluorescein azide isomer (5-FAM Azide) 0.1 mM was dissolved in 10 mM HEPES, pH 8.0. 5-FAM Azide was incubated with the cells for 16 h overnight at 4° C., washed thrice with PBS and imaged.

While the disclosure has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A reflectin polypeptide comprising an amino acid sequence that shares at least 70% sequence identity or at least 80% sequence homology with the amino acid sequence as set forth in SEQ ID NO:1, wherein said polypeptide substantially retains the activity of reflectin B1 (SEQ ID NO:1).

2. The reflectin polypeptide of claim 1, wherein the amino acid sequence shares at least 75%, at least 80, at least 85, at least 90, at least 91, 92, 93, 94, 95, 96, 97, 98, 99 or 100% sequence identity with the amino acid sequence as set forth in SEQ ID NO:1.

3. (canceled)

4. A nucleic acid molecule encoding the reflectin polypeptide according to claim 1.

5. The nucleic acid molecule of claim 4, wherein said nucleic acid molecule is comprised in a vector.

6. (canceled)

7. A host cell comprising the nucleic acid molecule of claim 4.

8. A recombinant reflectin nanoparticle, comprising the reflectin polypeptide of claim 1.

9. The recombinant reflectin nanoparticle of claim 8, wherein the reflectin polypeptide is conjugated to a ligand.

10. The recombinant reflectin nanoparticle of claim 9, wherein the ligand is covalently bonded to the reflectin polypeptide with a peptide bond.

11. The recombinant reflectin nanoparticle of claim 10, wherein the ligand has the following formula (I): 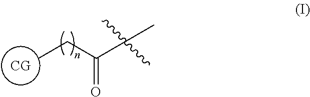
wherein
CG is a connecting group,
n is an integer selected from 1 to 5, and [symbol]
indicates the point of connection to a nitrogen atom of the reflectin polypeptide for formation of the peptide bond.

12. The recombinant reflectin nanoparticle of claim 11, wherein CG is a moiety of the following formula (II): 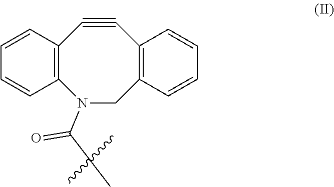 [SMILES: *C(=O)N1Cc2ccccc2C#Cc2ccccc21]
and [symbol] indicates the point of connection to the —(CH₂)ₙ moiety in the ligand of Formula (I).

13-15. (canceled)

16. The recombinant reflectin nanoparticle of claim 8, wherein the nanoparticle has a polydispersity index of below 0.5.

17-18. (canceled)

19. A method of synthesizing a recombinant reflectin nanoparticle of claim 8, the method comprising:
recombinantly expressing a reflectin polypeptide;
purifying the reflectin polypeptide by chromatography; and
self-assembling the purified reflectin polypeptide into a nanoparticle.

20-21. (canceled)

22. The method of claim 19, wherein the self-assembly is carried out in a buffer solution comprising an organic solvent.

23. The method of claim 22, wherein the concentration of the organic solvent determines a size of the recombinant reflectin nanoparticle.

24. The method of claim 23, wherein the organic solvent is acetonitrile.

25. (canceled)

26. A substrate surface-functionalized with a recombinant reflectin nanoparticle of claim 8.

27. The substrate of claim 26, wherein the recombinant reflectin nanoparticle is covalently immobilized on a surface of the substrate.

28-29. (canceled)

30. A method of immobilizing a recombinant reflectin nanoparticle of claim 8 on a substrate, the method comprising
providing a substrate comprising hydroxy groups;
reacting the hydroxy groups with a surface-bound spacer chain; and
providing the recombinant reflectin nanoparticle of claim 8 and reacting the recombinant reflectin nanoparticle with the surface-bound spacer chain.

31. (canceled)

32. The method of claim 30, wherein the recombinant reflectin nanoparticle comprises a triple bond functionality and the surface-bound spacer chain comprises an azide functionality and the method further comprises reacting the triple bond functionality with the azide functionality to form a covalent bond.

33. A skincare product comprising a recombinant reflectin nanoparticle of claim 8.

34. (canceled)
